(19) United States
(12) Patent Application Publication
(10) Pub. No.: US 2023/0211344 A1
(43) Pub. Date: Jul. 6, 2023

(54) HIGHLY SENSITIVE BIOMOLECULE DETECTION AND QUANTIFICATION

(71) Applicant: Cambridge Enterprise Limited, Cambridge (GB)

(72) Inventors: Tuomas Pertti Jonathan Knowles, Cambridge (GB); Kadi Liis Saar, Cambridge (GB); Georg Krainer, Cambridge (GB); William Emrys Arter, Cambridge (GB)

(73) Assignee: Cambridge Enterprise Limited, Cambridge (GB)

(21) Appl. No.: 17/909,026

(22) PCT Filed: Mar. 5, 2021
(86) PCT No.: PCT/EP2021/055614

§ 371 Date: Sep. 2, 2022

(30) Foreign Application Priority Data
Mar. 6, 2020 (GB) 2003324.7
May 20, 2020 (GB) 2007537.0

(51) Int. Cl. B01L 3/00; G01N 21/64

(52) U.S. Cl. CPC B01L 3/502761; G01N 21/6428; B01L 2200/16; B01L 2200/0652; B01L 2300/0636; B01L 2300/0645; G01N 2021/6439

(57) ABSTRACT

The present invention is directed to methods and devices capable of target analyte separation and analysis, in particular highly sensitive separation and detection and free-solution analyte detection assays.

### FIELD OF THE INVENTION

The present invention is directed to methods and devices capable of target analyte separation and analysis.

### BACKGROUND

A critical challenge in the context of biomolecular analysis and diagnostics is to detect analytes of interest at high specificity and high sensitivity.

To achieve this goal, many conventionally used biomolecular detection assays employ signal amplification strategies that selectively increase the concentration of targets of interest. For example, this is commonly achieved by polymerase chain reaction (PCR) for the detection of DNA based markers. However, in contrast to nucleic acids, protein based targets cannot be amplified directly, which has made their detection in biological settings more challenging.

To introduce specificity, protein detection and quantification assays commonly operate by surface-capturing target molecules of interest by suitable affinity reagents that allow their isolation prior to detection. Foremost amongst the techniques that employ this principle are enzyme-linked immunosorbent assays (ELISAs).

ELISA relies on immobilisation of the antigen of interest. This can be accomplished either by direct adsorption to the assay plate or indirectly via a capture antibody that has been attached to the plate. The antigen is then detected either directly via a labelled primary antibody or indirectly via a labelled secondary antibody. The most powerful ELISA assay is a “sandwich” capture assay, so called because the analyte to be measured is bound between two primary antibodies, the capture antibody and the detection antibody.

Direct ELISA involves a labelled primary antibody that reacts directly with the antigen. Advantages of direct ELISA are that the process is quicker since it uses one detection antibody and fewer steps, and that cross-reactivity of a secondary detection antibody is avoided. However, direct ELISA is not commonly used because there is minimal signal amplification.

More common is indirect ELISA (particularly sandwich capture ELISA), which uses a labelled secondary antibody for detection, and is the most popular format for ELISA. The secondary antibody has specificity for the primary antibody. In a sandwich ELISA, it is critical that the secondary antibody is specific for the detection primary antibody only (and not the capture antibody) or the assay will not be specific for the antigen. Generally, this is achieved by using capture and primary antibodies from different host species (e.g., mouse IgG and rabbit IgG, respectively). For sandwich assays, it is beneficial to use secondary antibodies that have been cross-adsorbed to remove any secondary antibodies that might have affinity for the capture antibody. Having multiple epitopes on the primary antibody allows multiple secondary detection antibodies to bind, thereby allowing for signal amplification and increased sensitivity. Sandwich assays therefore enable enzyme-driven signal amplification. However, sandwich assays have drawbacks since cross-reactivity might occur with the secondary antibody, resulting in nonspecific signal, and an extra incubation step is required in the procedure.

ELISA techniques have been developed to improve sensitivity and throughput, including replacing the requirement for the enzymatic signal amplification step. These include immuno-PCR, plasmonic-ELISA and magnetic and electrochemical readouts of the ELISA signal. However, due to the surface-based nature of ELISA, these approaches nevertheless remain prone to false-positive signals due to non-specific surface-binding of non-target species. Moreover, the multiple washing and blocking steps required to minimise this effect are time-intensive and limit the assay throughput while the requirement for multiple antibodies introduces the possibility of cross-reactivity. Furthermore, sandwich ELISAs are not possible for monoepitopic proteins, or for targets that lack an antibody pair that works well in a sandwich format.

Recently, a number of techniques have emerged that enable enhanced detection sensitivity and much more extensive multiplexing of protein targets than has been possible by conventional ELISAs. In this context, bead-based ELISAs, for instance, allow improved sensitivity and automation of the assay workflow. Additionally, bead-based flow-cytometry methods as well as assays that aim to exploit DNA barcoding for massively parallel biomolecular sensing, have shown remarkable sensitivity for the detection of a wide variety of targets. However, these approaches similarly retain the requirement for 1) well-validated antibody pairs, and, crucially, rely on 2) multi-step protocols and require 3) surface-immobilisation of the target protein.

Recently, droplet based digital detection has been developed using highly parallel microfluidic droplets for single enzyme molecular detection. Although this provides in-solution detection without washing steps, it requires proximity probes (for example fluorophores generating FRET signals, oligonucleotide sequences hybridising to each other) coming to close proximity and generating a signal only in the presence of the protein molecules. The methods are also complex in terms of sample partitioning and detection.

As such, there remains a need for detection techniques with improved sensitivity, specificity and/or throughput and ease of use.

### SUMMARY OF THE INVENTION

Accordingly, in a first aspect of the present invention there is provided a microfluidic device for investigating a target biomolecule comprising:
a) a separation region configured to separate said target biomolecule from a fluid sample comprising a heterogeneous mixture of material; and
b) a detection region configured for highly sensitive of said target biomolecule.

In a further aspect, there is provided a microfluidic device for investigating a target biomolecule comprising:
a) a separation region configured to separate said target biomolecule from a fluid sample comprising a heterogeneous mixture of material; and
b) a detection region configured for highly sensitive detection of said target biomolecule;
for use in a method of detecting a biomarker useful in the clinical diagnosis of a disease. The biomolecule being a biomarker may be a biomarker for any disease including cancer, neurodegenerative diseases, infectious diseases including viral, bacterial or other pathogens, or cardiovascular diseases.

In a further aspect there is provided a method of investigating a target biomolecule comprising:
a) introducing a fluid sample comprising a heterogeneous mixture of material including a target biomolecule into a microfluidic device as defined herein;
b) separating said target biomolecule from said heterogeneous mixture in said separation region; and
c) performing detection on said target biomolecule in said detection region.

The present invention enables target analyte separation and digital detection and provides for a highly sensitive assay. By combining rapid separation of target analyte(s) with simultaneous digital detection via a single solution-phase system gives the present invention a number of advantages over existing technologies. The present invention provides a highly sensitive biomolecule detection and quantification strategy. In addition to providing a very high level of sensitivity down to sub-picomolar levels, the present invention is implemented in a surface-immobilisation free manner and can thus be used for performing biomolecule detection assays in a single step in contrast to incumbent assays that rely on surface-mobilisation and an array of washing steps.

Avoiding surface-immobilisation and using solution based processing enables immuno-sensing possibilities for mono-epitopic targets. It also opens up the possibility to separate and analyse different physical forms of the same target (for example aggregated vs non-aggregated proteins). The present invention can also infer concentrations without calibration. The speed of the present invention vs existing technologies (seconds vs hours) provides significant process advantages.

The present invention differs from conventional immunosorbent protein detection techniques in that immunosorbent assays are: surface based, multi-step assay, specificity from affinity criterion only; require an affinity reagent pair. In contrast, the digital detection techniques according to the present invention may be: in free solution; a single step assay; have specificity from combined affinity and electrophoretic criteria; and can be single affinity reagent. The surface-free nature of the invention can reduce non-specific electrostatic binding events, but also gives the basis for the assay to be operated without the requirement for a multistep protocol.

In a further aspect there is provided a method of investigating a target biomolecule comprising:
a) adding a suitable affinity reagent to a solution comprising a target biomolecule to form a fluid sample comprising bound affinity reagent-target biomolecule and unbound affinity reagent;
b) introducing said fluid sample into a microfluidic device as defined herein (or said affinity reagent can be added to said solution comprising a target biomolecule within the microfluidic device);
c) separating said bound affinity reagent-target biomolecule from unbound affinity reagent target biomolecule in said separation region; and
d) performing highly sensitive, such as single molecule counting or digital, detection on said bound affinity reagent-target biomolecule in said detection region.

Due to in-solution target biomolecule capture and fast removal of excess affinity reagent, high concentrations of the affinity reagent can be employed. Furthermore, affinity reagents with a relatively low binding constant can be used. This enables quantitative target biomolecule-capture regardless of affinity reagent affinity and further allows the affinity reagent-target biomolecule binding interaction to be maintained during the entire sensing process.

In a further aspect, there is provided a method of investigating a target biomolecule comprising:
a) introducing a fluid sample comprising a heterogeneous mixture of material including a target biomolecule into a microfluidic device as defined herein;
b) separating said target biomolecule from said heterogeneous mixture in said separation region; and
c) performing detection on said target biomolecule in said detection region.

In a further aspect, there is provided a method of investigating a target biomolecule, wherein said target biomolecule is complexed with an affinity reagent; said method comprising introducing a fluid sample comprising a heterogeneous mixture of material including said target biomolecule and affinity reagent into a microfluidic device; separating said target biomolecule from said heterogeneous mixture in a separation region; and detecting said target biomolecule; wherein said target molecule separation step is based on properties associated with the target biomolecule.

In a further aspect, there is provided a method of detecting multiple biomolecules simultaneously comprising:
a) incubating one or more affinity reagents with a target biomolecule to form target biomolecule bound affinity reagent(s);
b) separating said target biomolecule bound affinity reagent(s) from unbound affinity reagent and optionally other material; and
c) detecting one or more properties of said target biomolecule bound affinity reagent(s);
wherein the method is performed using a microfluidic device as described herein.

In a further aspect, there is provided a method for detecting a target biomolecule (biomarker), including but not limited to cancer biomarkers, neurodegenerative disease biomarkers, infectious disease biomarkers including viral, bacterial or other pathogens, or cardiovascular disease biomarkers (including fibrils or smaller oligomers), performed on a sample from a subject, including for example a blood, brain or other sample, said method comprising:
a) optionally carrying out processing steps on said subject sample to obtain a fluid sample;
b) adding a suitable affinity reagent to said fluid sample to form a fluid sample comprising bound affinity reagent-target biomolecule and unbound affinity reagent;
c) introducing said fluid sample into a microfluidic device as defined herein (or said affinity reagent can be added to said solution comprising a target biomolecule within the microfluidic device);
d) separating said bound affinity reagent-target biomolecule from unbound affinity reagent target biomolecule in said separation region;
e) detecting said bound affinity reagent-target biomolecule in said detection region such that detection of said target biomolecule according to clinically relevant parameters indicates presence of such disease.

In a further aspect, there is provided an in vitro method for identifying an individual having risk of disease, including but not limited to cancer, neurodegenerative diseases, infectious diseases including viral, bacterial or other pathogens, or cardiovascular disease, comprising:
a) optionally carrying out processing steps on a sample from a subject sample to obtain a fluid sample;
b) adding a suitable affinity reagent to said fluid sample to form a fluid sample comprising bound affinity reagent-target biomolecule and unbound affinity reagent;
c) introducing said fluid sample into a microfluidic device as defined herein (or said affinity reagent can be added to said solution comprising a target biomolecule within the microfluidic device);
d) separating said bound affinity reagent-target biomolecule from unbound affinity reagent target biomolecule in said separation region;
e) detecting said bound affinity reagent-target biomolecule in said detection region;
to detect an individual having risk of said disease.

In a further aspect, there is provided a method for diagnosing a disease (including but not limited to detecting cancer, neurodegenerative diseases, infectious diseases including viral, bacterial or other pathogens, or cardiovascular disease) or an increased risk of having said disease in a subject, comprising the step of using a device according to the present invention or a method according to the present invention to measure a biomarker in a biological sample isolated from said subject.

### BRIEF DESCRIPTION OF THE FIGURES

FIG. 1 shows an exemplary device according to aspects of the present invention. (a) shows a sample, including a mixture of the target protein and its fluorescently labelled probe, injected into a micron scale electrophoretic separation unit illustrated in panel (b). The application of electric field allows protein-bound probe molecules to be discriminated from those probe molecules that are not bound to the protein target, owing to a difference in their electrophoretic mobilities. (c) The protein concentration is determined using a single-molecule confocal spectroscopy setup that screens the cross-section of the device and thereby allows the flux of the protein bound probe molecules to be estimated. (d) shows conventional ELISAs (and their adaptions) vs the technique of the present invention. (e) demonstrates the principle of detection/sensing of biomolecular binding reactions and immuno-complexes.

FIG. 2 shows exemplary physico-separation based on affinity reagents. The Figure shows the use of optional additional linkers.

FIG. 3 shows an example of field mediated separation of bound and unbound material, (a) as part of a continuous flow with a field perpendicular to the flow, and (b) under batch separation with a field parallel to the flow.

FIG. 4 shows an exemplary detection region providing single molecule detection.

FIG. 5 shows analysis of αS oligomers using a device made according to the present invention. FIG. 5a shows an electropherogram of synthetic αS oligomer at 0 V (dark green, blue line) and 300 V (light green, red line) acquired using scanning mode. The coloured sections in FIG. 5a correspond to time traces of same colour in FIG. 5(b). FIG. 5b (top panel) shows time traces of fluorescence bursts gathered in stepping mode, for positions in electropherogram colour-coded in (a); and FIG. 5(b) (bottom panel) shows histograms of single-burst fluorescence intensity corresponding to the time traces and electropherogram positions shown in (b).

FIG. 6 shows analysis of the size-mobility relationship for αS oligomers.

FIG. 7a shows that binding of monovalent streptavidin to a biotinylated DNA sequence reduces the electrophoretic mobility of the latter species;

FIG. 7b shows an electrophoretogram across the cross-section of the channel for the mixture (red line) and for a control sample (blue line) at the mid-height of the channel, demonstrating the presence of both streptavidin bound and non-bound biotin molecules in the sample. The elution regions shaded in light grey were used to extract the number of streptavidin-biotin complexes that passed the device in a given time, and ultimately, their concentration.

FIG. 7c shows photon count time traces for the mixture (left) and the control sample (right) at the position where the concentration of the complex molecules was the highest (dark grey region in panel (b)).

FIG. 8 shows detection of IgE. FIG. 8a shows binding of the aptamer probe to its target species (IgE) reduces the electrophoretic mobility of the probe, setting the basis for removing the excess probe molecules electrophoretically. FIG. 8b shows electrophoretogram across the cross-section of the channel was recorded for the sample (redline) and for the free probe (blue line) to estimate the concentration of the IgE molecules in the sample. The concentration was estimated by monitoring the flux of the fluorescent molecules in the elution region shaded in light grey. FIG. 8c shows photon count time traces for the mixture (left) and the control sample (right) at the position where the concentration of the complex molecules was the highest (dark grey region in panel (b)). The time traces across the full elution region yielded a concentration estimate of 21.7 pM.

FIG. 9 shows how free solution immunosensing can overcome fundamental limitations of surface-based sensing methods. FIG. 9a shows a schematic of surface-based immunosensor assays (e.g., ELISAs or bead-based assays) and their inherent limitations in terms of capture efficiency, analyte dissociation, and workflow complexity. Conventional methods are limited to surface-capture probe concentrations (c_{probe}) in the low nanomolar regime (1-2 nM). Under these conditions, a significant amount of the analyte is not bound and thus remains undetected, especially when using affinity probes with K_{d} > 1 nM (see panel b(i)). Additionally, the binding equilibrium is disturbed during washing steps that take tens of minutes to several hours. Hence, dissociation of the immunoprobe-analyte complex becomes significant, particularly for weak-binding affinity probes with K_{d} > 1 nM (see panel b(ii)). Moreover, conventional assays involve multi-step procedures and typically require additional affinity probes for detection. FIG. 9b shows speciation curves depicting the fraction of probe-bound analyte vs affinity probe concentration (panel b(i)) and fraction of probe-bound analyte vs time (panel b(ii)). Shown are simulations for probe-analyte affinities with K_{d} = 0.1-1000 nM (from light to dark blue). Green and red shaded areas denote operation regimes. In panel b(ii), t = 0 refers to the moment when the unbound probe is removed from the system. FIG. 9c shows a schematic illustration depicting the principles of a free-solution immunosensor assay and its advantages over conventional surface-based methods. By performing the immunosensor reaction in solution, arbitrarily high concentrations of the affinity probe can be used, which permits quantitative antigen binding, even for affinity reagents with K_{d} > 1 nM (see panel b(i)). A rapid timescale for the removal of non-target bound probe prevents the system from re-equilibrating (see panel b(ii)) and sets the basis for quantitative analysis of the immunoprobe-analyte complex interaction. Additionally, the assay can be accomplished in a single step and requires only a single affinity reagent.

FIG. 10 shows the detection of weak biomolecular binding interactions on the example of α -synuclein fibrils and their aptamer probe. FIG. 10a shows the binding of the aptamer probe to α-synuclein fibrils reduces the electrophoretic mobility of the probe, allowing for discrimination between probe-bound and unbound species. FIG. 10b shows an electropherogram (left panel) as obtained by stepwise scanning of the confocal volume across the cross-section of the channel for the α-synuclein fibrils-aptamer sample (blue line; average of n = 3 repeats, the shaded bands correspond to the standard deviation) and for the free aptamer probe (purple line). The right panel shows a zoom-in region of the electropherogram. The shaded region in grey where the concentration of the complex exceeded that of the free probe (1150 µm < x < 2000 µm) was used to estimate the concentration of the fibrils. Note: the photon arrival frequency in the region where x < 1100 µm (i.e., where the probe elutes) was too high to count molecules one-by-one, hence the detected number of molecules in this region should be viewed as an approximation. This could be overcome through calibration in this region or to avoid quantitative analysis in this region. FIG. 10c shows exemplary photon-count time traces for the control sample (left panel, purple) and the mixture (right panel, blue) at the position indicated with coloured dots in panel b rhs. The number of molecules at each of the scanned positions was estimated using a burst-search algorithm as detailed in the methodology section.

FIG. 11 shows label-free detection of single protein molecules and protein assemblies using interferometric scattering (iSCAT) microscope.

### DETAILED DESCRIPTION OF THE INVENTION

The work leading to this invention has received funding from the European Research Council under the European Union’s Seventh Framework Programme (FP7/2007-2013) / ERC grant agreement no337969.

The project leading to this application has received funding from the European Union’s Horizon 2020 research and innovation programme under the Marie Sklodowska-Curie grant agreement No 841466.

The invention will now be described with reference to the following non-limiting examples. The following embodiments apply to all aspects of the present invention.

The present invention describes methods and devices that use a solution based system to enable rapid separation of target analyte(s) with simultaneous ‘single molecule’ detection. The system makes use of microfluidic device technology. Assays performed according to the present invention may be considered direct digital immunosensor assays.

In an embodiment, in order to investigate target biomolecule(s), the present invention introduces a fluid sample, comprising a heterogeneous mixture of material including a target biomolecule, into a microfluidic device comprising a separation region and a detection region. The method firstly involves separating the target biomolecule and then detects one or more properties of said target biomolecule on a single molecule basis.

The present invention enables analysis of a “target biomolecule”. The target biomolecule may be considered the analyte. The invention is not particularly limited by the type of biomolecules it can identify. Suitable biomolecules include proteins, peptides, modified peptides (including post-translational and chemical labelling modifications), antibodies, amino acid conjugates of non-proteinaceous nature, non-biological amino acid containing proteins and peptides or amino acid conjugates and the like, protein complexes, clusters and aggregates, and phase-separated protein condensates. The target biomolecule may be a bimolecular complex, or macromolecular complex. Examples of biomolecular complexes include: protein complexes, such as multienzyme complexes, including proteasome, DNA polymerase III holoenzyme, RNA polymerase II holoenzyme, symmetric viral capsids, chaperonin complex GroEL-GroES, photosystem I, ATP synthase, ferritin; RNA-protein complexes including ribosome, spliceosome, vault, SnRNP, ribonucleoproteins (RNPs); DNA-protein complexes, including nucleosome; and protein-lipid complexes, including lipoprotein. In one example, the target biomolecule is a biomarker. The biomarker may take any of the forms described above.

Target biomolecules according to the present invention can include monoepitopic targets, which cannot be detected by current sandwich-ELISA techniques. Target biomolecules may include transient species.

The present invention enables the detection of biomolecules that are challenging to detect with current approaches. This includes, for example, intrinsically disordered proteins, prions, isoforms of the same protein, oligomeric protein clusters or aggregates and phase-separated protein condensates. In particular, it is known that a number of aggregate-prone proteins such as tau, alpha-synuclein and huntingtin mis-fold into smaller oligomers before forming large aggregates or fibrils. While the presence of oligomers is transient, it is believed that these are the most cytotoxic species. The versatility of the present invention enables detection of hitherto challenging biomolecules.

The present invention may detect a single species of target biomolecule. Alternatively, the present invention may detect multiple target biomolecules in parallel and is therefore suitable for multiplexing applications. This can be achieved in various ways, including by selecting probes that can bind to multiple targets in same mixture and then employing separation and detection in each region of interest to monitor multiple species. It is also possible to include multiplexing with multiple wavelengths when undergoing detection. As such, the present invention encompasses both detection of a single species or multiple species within the same analysis.

The target biomolecules will typically be in a fluid sample comprising a heterogeneous mixture of material that must be separated before detection. The term “heterogeneous mixture of material” means a solution generally containing one or more target biomolecule(s) and additional material. This additional material may be any material, for example: non-target biomolecules, for example proteins and the like; unreacted material within the analyte solution for example unbound affinity probes; or any other material that is desired to be excluded from the subsequent analysis step.

The “fluid sample” is a solvent system containing the target biomolecules. Any suitable solvent system is contemplated within the present invention. Suitable solvent systems will preferably be compatible with the device. Suitable solvent systems will preferably be compatible with the target biomolecule. Suitable solvent systems may facilitate separation and/or detection. The skilled person will select suitable solvent(s) according to the separation and/or detection methods used as well as the target biomolecules. Suitable solvent systems include (but are not limited to) any aqueous solution or buffer, for example phosphate buffer, phosphate-buffered saline, tris-HCl, HEPES, acetate buffer, borate buffer and the like.

The fluid sample containing the target biomolecules may be processed prior to introduction into the device. This may include a simple incubation step or a more complex array of steps, such as incubation followed by off-chip separation and/or off-chip washing to reduce the complexity of the mixture prior its injection. However, in some embodiments, the fluid sample can be directly introduced into the device with no or minimal processing.

The present invention makes use of microfluidic device technology. “Microfluidic device” is intended to be interpreted broadly and encompasses any suitable small volume device. Suitable microfluidic devices may be made via any known techniques including lithography but can also encompass devices made using injection moulding, 3D printing and the like. Microfluidic devices may be termed “chips”. In an embodiment, the microfluidic device has a volume of between 10⁻⁴ mm³ to 10 mm³.

Suitable devices according to the present invention include a separation region to separate the target biomolecule and a detection region to measure one or more properties of the target biomolecule on a single-molecule basis. These regions may be spatially separated. Alternatively, the separation region and detection region may overlap to a lesser or greater extent. For example, it is possible for the target biomolecules to be still undergoing separation while they are being detected, provided sufficient separation has taken place to resolve the target biomolecule(s) of interest.

In an embodiment, the separation and detection are undertaken on a single chip. Alternatively, the device may comprise a number of modular sub-elements to facilitate selection of different separation and/or detection techniques depending on the particular needs. This allows for a configurable system that is efficient and flexible, whereby the user can select particular separation and/or detection techniques as required. With a modular system, however, the modular elements are in fluid communication so that the separation and detection steps are a single overall process.

The device comprises a “separation region”. The separation region is designed to separate target biomolecules from other material within the fluid sample. Any suitable separation technique available within a microfluidic device is encompassed by the present invention. Exemplary separation processes include electrophoresis, both free-flow and capillary; diffusion; isoelectric separation (isoelectric focussing); chemical separation; sizing based separation, dielectrophoresis, diffusiophoresis, thermophoresis, isotactophoresis and the like. As can be seen, in a broad embodiment, any suitable on-chip (i.e. within the device) separation technique is encompassed. Separation may be carried out in continuous (free-flow) or non-continuous (batch) modes. As such, the separation process may be performed in the direction of the flow (e.g. non-continuous or batch separation with techniques such as capillary electrophoresis) or orthogonally to the flow (e.g. continuous flow process, with techniques such as free flow electrophoresis). The present invention encompasses both parallel or orthogonal separation techniques. In an embodiment, separation is via a parallel flow technique. In an alternative embodiment, separation is via technique orthogonal to the flow.

Separation may be undertaken via a field-mediated approach. By “field-mediated”, it is meant that a field is applied to the fluid sample which has the effect of separating the target biomolecule(s) from other material.

The present invention utilises differences in the properties of the target biomolecule itself to enable separation with in-situ detection.

The separation region converts a heterogeneous mixture of material within a fluid sample into a (at least more) homogeneous sample. This spatially separates the target biomolecule such that detection can be focused on the region where the target biomolecule is found. This may involve only directing detection to the region in question or focusing on the region in question during post-analysis.

In a preferred embodiment, the separation technique is separation based on electrophoretic mobility. This intrinsic physical property is related to the biomolecules’ ratio of net electrical charge to size.

As outlined above, separation may include separation of transient species. An example is the separation of oligomers from monomers. Such oligomers may not be easily detectable via standard techniques since they are only present transiently. A specific example is the detection of oligomeric α-synuclein (αS) which is important for the investigation of Parkinson’s disease but is currently challenging to detect since the oligomers typically exist only transiently as highly heterogeneous mixtures present at low concentrations.

The separation region may preferably resolve heterogeneous protein mixtures. The separation region may preferably resolve immuno-complexes from unbound immuno-probes. The separation region may preferably separate physical properties of otherwise similar biomolecules, for example aggregated vs non-aggregated proteins.

The device further comprises a “detection region”. This is a region of the device in which the target biomolecule is investigated on a digital basis to determine one or more properties. The detection region is therefore configured to enable digital detection. By digital detection, it is meant that the system performs measurements on a single-molecule basis. Such an approach enables high sensitivity and specificity.

By “digital basis” or “digital detection” it is intended to mean that properties of the biomolecule are assessed on a single-molecule basis rather than in bulk. An example of digital detection is counting individual biomolecules.

“Single molecule” is intended to encompass single-complex or single-molecule resolution. A complex may be the natural conformational state of the biomolecule (for example an oligomer) or may be the target biomolecule complexed with the probe.

Digital detection may be considered “highly sensitive” detection that is more sensitive than detection in bulk.

Devices and methods according to the present invention may therefore utilise highly sensitive detection. In embodiments, highly sensitive detection may be considered detection at a sensitivity level greater than bulk detection. In embodiments, highly sensitive detection may be detection of low concentration biomolecules. Low concentration in embodiments may be nanomolar. Low concentration in embodiments may be picomolar. Low concentration in embodiments may be sub-picomolar. In alternative embodiments, highly sensitive detection may be single molecule detection. As said above, single molecule is intended to encompass single-complex or single-molecule resolution. Thus, the present invention allows for single molecule detection but also detection at a less sensitive level than single molecule detection but still at a sensitivity level far greater than bulk detection. In embodiments, the detection technique uses single molecule detection equipment capable of detecting individual events. In some embodiments, the single molecule detector operates in a way that resolves individual biomolecules (events). However, in other embodiments, the single molecule detector operates in a way where individual events are not resolvable, for example due to higher sample concentration. Thus, in embodiments, the single molecule detector may be used in a mode which no longer resolves individual events. This is different to current bulk detection techniques.

In an embodiment, detection is carried out wherein the target biomolecule(s) is present at a concentration of less than 10,000 nM, less than 9000 nM, less than 8000 nM, less than 7000 nM, less than 6000 nM, less than 5000 nM, less than 4000 nM, less than 3000 nM, less than 2000 nM, less than 1500 nM, less than 1000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 9 nM, less than 8 nM, less than 7 nM, less than 6 nM, less than 5 nM, less than 4 nM, less than 3 nM, less than 2 nM, or less than 1 nM.

In an embodiment, detection is carried out wherein the target biomolecule(s) is present at a concentration of less than 10,000 pM, less than 9000 pM, less than 8000 pM, less than 7000 pM, less than 6000 pM, less than 5000 pM, less than 4000 pM, less than 3000 pM, less than 2000 pM, less than 1500 pM, less than 1000 pM, less than 900 pM, less than 800 pM, less than 700 pM, less than 600 pM, less than 500 pM, less than 400 pM, less than 300 pM, less than 200 pM, less than 100 pM, less than 90 pM, less than 80 pM, less than 70 pM, less than 60 pM, less than 50 pM, less than 40 pM, less than 30 pM, less than 20 pM, less than 10 pM, less than 9 pM, less than 8 pM, less than 7 pM, less than 6 pM, less than 5 pM, less than 4 pM, less than 3 pM, less than 2 pM, or less than 1 pM.

Any suitable detection technique is encompassed by the present invention. Suitable detection techniques include optical detection, for example fluorescence spectroscopy, including confocal microscopy. Other suitable techniques include other optical detection techniques such as TIRF microscopy or iSCAT.

Suitable optical detection may be single wavelength or multi-wavelength. In a particular embodiment, both single-wavelength or multi-wavelength confocal microscopy is envisaged by the present invention. Using multi-wavelength strategies enables the detection of multiple targets and/or multiple properties at the same time.

Alternatively, the techniques may be non-optical. This may be, for example conductance based sensing such as nanopore. Other single molecule detection approaches are encompassed by the present application. The detection region may measure other properties of the target biomolecule. The detection region may count target biomolecules.

It is also possible to infer properties of the target biomolecules from other aspects of the device. For example, measuring the potential applied to the device and understanding the device geometry enables the inference of properties such as the electrophoretic mobility of the analyte molecules. Thus through careful control and monitoring of the device properties, additional information can be gained from the target biomolecules. Such information is not limited to being obtained from the detection region. In embodiments of the invention, the target biomolecule is investigated in regions outside of the detection region.

The target biomolecules may be unlabelled or labelled to facilitate detection depending on particular needs.

By way of example, fluorescence is inherent in and detected from certain amino acids directly (for example tryptophan, tyrosine, and phenylalanine) and can be analysed directly using fluorescence spectroscopy. However, in a preferred embodiment, labelling with a fluorophore is used.

In an embodiment, affinity reagent(s) may be used to facilitate separation and/or detection. Suitable affinity reagents include, but are not limited to: nucleic acids, polypeptides and peptides and fragments thereof, ribonucleoproteins, a protein-nucleic acid complex, antibodies, antibody fragments, antigen binding antibody fragments, nanoparticles, nanobodies, viruses or viral-like particles, enzymes, aptamers, affimers and other non-antibody binding proteins/molecules. The affinity reagent can be labelled or unlabelled. Examples of suitable labels include fluorophores and radioactive labels.

Suitable affinity reagents include nucleotide moieties. Preferably, the nucleotide moiety is an oligonucleotide, such as DNA oligonucleotide or RNA oligonucleotide. The moiety may be a DNA-aptamer or RNA-aptamer. The affinity reagent may be a complex, for example a polypeptide/ oligonucleotide complex such as an antibody conjugated to a DNA/RNA sequence.

Thus, the present invention comprises affinity reagents as described herein further comprising a nucleotide moiety.

Suitable oligonucleotide will be determined by the skilled person, but exemplary lengths include between 10-100 bp.

Aptamers are considered an attractive class of affinity reagents because while offering recognition capabilities that rival those of antibodies, they can readily be produced by chemical synthesis and they elicit little or no immune response in therapeutic applications. Moreover, their relatively small physical size in comparison to full antibodies permits a more significant alteration in its electrophoretic mobility upon binding to a target (if this is the separation technique used), setting the basis for an efficient electrophoretic separation between the protein bound and non-bound forms of the molecule. Further, aptamer affinity reagents are known to be relatively homogenous, which ensures that the unbound reagent elutes at a well-defined position - a characteristic that may be harder to achieve for antibodies, especially if their binding is not residue specific.

The present invention provides new methodologies using affinity reagents (probes). The present invention enables methods of investigating a target biomolecule comprising:
a) adding a suitable affinity reagent to a solution comprising a target biomolecule to form a fluid sample comprising bound affinity reagent-target biomolecule and unbound affinity reagent;
b) introducing said fluid sample into a microfluidic device as defined herein (or said affinity reagent can be added to said solution comprising a target biomolecule within the microfluidic device);
c) separating said bound affinity reagent-target biomolecule from unbound affinity reagent target biomolecule in said separation region; and
d) performing digital detection on said bound affinity reagent-target biomolecule in said detection region.

Step b) introduces the fluid sample into the microfluidic device after the affinity reagent has been added. It is equally possible to incorporate this step into the microfluidic device itself such that a fluid sample comprising the target biomolecule is introduced into the device whereafter a suitable affinity reagent is added.

These new methodologies are solution-based methodologies.

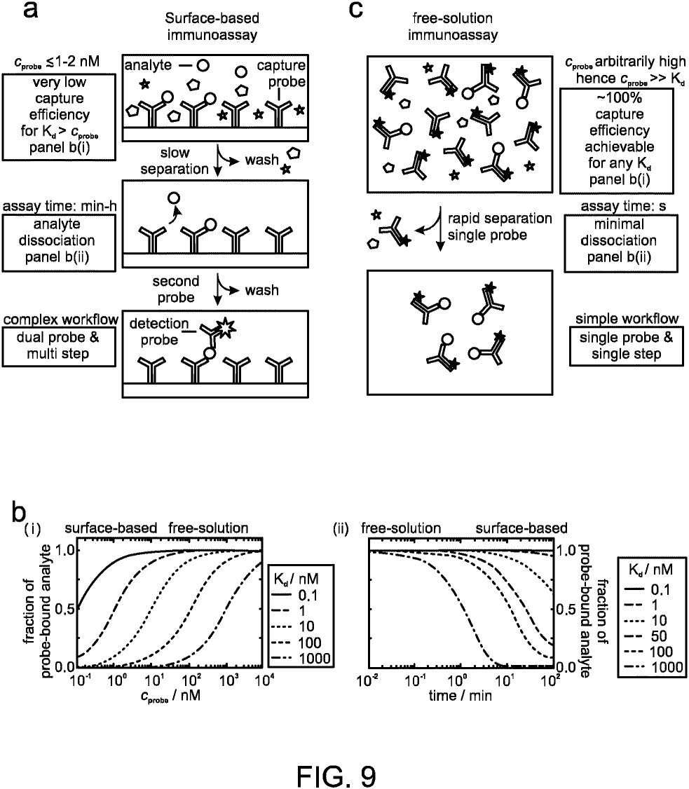

The advantages of the present invention over conventional ELISA techniques are further shown in FIG. 9. Conventional sensing approaches remain reliant on surface-immobilization of target analyte molecules via capture probes, and multi-step washing protocols for the removal of detection antibodies and reagents. These features constrain parameter space in assay design, resulting in fundamental limits in assay sensitivity due to the underlying thermodynamics and kinetics of the immunoprobe-analyte interaction (FIG. 9a). Principally, the concentration of affinity capture probes that can be utilized in such approaches is limited by the finite area of the surface. The maximally achievable surface-capture probe concentrations in surface-based methods is ∼1-2 nM. As such, for a capture probe with a dissociation constant (K_{d}) of 1 nM, as is typical of many affinity reagents, only 50 % of an analyte will be bound to the probe, which falls to <1 % for capture probes with K_{d} > 100 nM (FIG. 9a,b(i)). This low capture efficiency is exacerbated by dissociation of the probe-analyte complex (particularly for K_{d} > 1 nM) as the binding equilibrium is disturbed over tens of minutes to hours by the washing steps required by the ELISA or bead-based protein detection assays; hence, off-rates become significant (FIG. 9a,b(ii)). These lengthy assay workflows thus present challenges in maintaining probe-analyte interactions under non-equilibrium conditions. Together, these effects constitute intrinsic drawbacks to surface-based methods, resulting in the limitation that highly optimized capture probes with sub-nanomolar dissociation constants are required for effective sensing, and that a calibration step is needed that converts the recorded signal to the actual target concentration.

The present invention can overcome these drawbacks through two distinguishing elements.

Firstly, analyte capture can be conducted in the presence of arbitrarily high concentrations of the affinity capture probe. This feature ensures near-quantitative binding of the target molecule (FIGS. 9b,c), and can be achieved, for instance, by performing the assay in solution.

The concentration of affinity reagent can be selected by considering the anticipated analyte concentration and dissociation constant K_{d} of the probe. The concentration can be increased as required to ensure complete (quantitative) or near complete binding. In contrast to current techniques, concentration may be in the mid to high nanomolar range, the micromolar or millimolar range.

In an embodiment, arbitrarily high concentrations of affinity probe is used. Arbitrarily high means an excess of probe is used vs the target analyte to achieve the advantages discussed herein.

In an embodiment, the probe concentration used in the present invention may be greater than 2 nM, greater than 5 nM, greater than 10 nM, greater than 50 nM, greater than 100 nM, greater than 200 nM, greater than 500 nM, greater than 1 µM, greater than 10 nM, greater than 1 µM, greater than 10 µM, greater than 100 µM, greater than 500 µM, greater than 1 mM, greater than 10 mM, greater than 100 mM, or greater than 500 mM. Significantly higher probe concentrations are possible with the present invention as compared against traditional detection techniques.

In an embodiment, the probe used in the present invention has a dissociation constant K_{d} of 1 nM or more, 5 nM or more, 10 nM or more, 100 nM or more, 1000 nM or more, or more. It is possible to make use of probes with significantly higher dissociation constants than has been possible with traditional detection techniques. This opens the possibility to use different probes than currently possible opening the potential for new analysis techniques.

Secondly, excess (unbound) probe removal is suitably fast (i.e., on timescales much faster than the half-time of probe-analyte dissociation). In this way, sensing can take place before the system re-equilibrates and the probe-analyte complex dissociates (FIG. 9b and FIG. 9c). In an embodiment, unbound probe is substantially separated from the bound probe (such that detection of the bound analyte can be undertaken) in less than 5 minutes, less than 1 minute, less than 30 seconds, less than 15 seconds, less than 10 seconds, less than 5 seconds or on the order of 1-2 seconds. This timeframe is significantly lower than the excess probe removal steps based on traditional detection techniques. The separation timeframe is lower than the half-time of probe-analyte dissociation time.

Together the possibility to use arbitrarily high probe concentrations in solution, and to remove unbound probe in a rapid manner, overcomes fundamental drawbacks of incumbent surface-based sensing approaches.

By performing the immunosensor reaction in solution, arbitrarily high concentrations of the affinity probe can be used, which permits quantitative antigen binding, even for affinity reagents with K_{d} > 1 nM (see panel b(i)). A rapid timescale for the removal of non-target bound probe prevents the system from re-equilibrating (see panel b(ii)) and sets the basis for quantitative analysis of the immunoprobe-analyte complex interaction. Additionally, the assay can be accomplished in a single step and requires only a single affinity reagent.

The present invention allows for the immunosensor reaction to be carried out in solution. This solution based approach provides advantages over the prior methods. The present invention allows arbitrarily high concentrations of affinity probe to be used, which permits quantitative antigen binding, even for affinity reagents with K_{d} > 1 nM (see FIG. 9b(i)). The present invention allows a rapid timescale for the removal of non-target bound probe which prevents the system from re-equilibrating (see FIG. 9b(ii)). This enables the present invention to achieve quantitative analysis of the immunoprobe-analyte complex interactions. The present invention also allows the assay to be accomplished in a single step. The present invention also requires only a single affinity reagent.

The present invention operates in free solution and allows for separation of excess probe without the need for washing steps. As outlined above, the ability to use arbitrarily high concentrations of binding probes enables the probe-target binding equilibrium to be favourably manipulated to optimize target capture efficiency (see (FIGS. 9b,c). Furthermore, the assay operates on a fast timescale (for example ~2 s), meaning that the probe-analyte binding interaction is maintained during the entire sensing process (FIGS. 9b,c). Combined, these factors allow the use of relatively weak- binding capture reagents (for example Kd = 10-1000 nM), whereas conventional approaches require highly-optimized probes with sub-nanomolar affinity.

Avoiding the need for highly-optimized probes (for example ultra-high-affinity antibody probes) offers significant advantages over prior separation techniques. Generating such highly-optimized probes is a costly and non-trivial process, and may not be feasible for some biomarker targets, such as highly heterogeneous biomolecular complexes, including protein aggregation species. The present invention opens up the possibility to use commonly overlooked affinity reagents, such as aptamers, in quantitative single-molecule sensing.

In an embodiment, there is provided a method of quantitative single-molecule sensing using aptamers. Such an approach has hitherto not been possible.

Furthermore, by incorporating the digital detection techniques of the present invention together with affinity selection, the present invention achieves selectivity in analyte (for example protein) sensing.

The surface-free nature of the assay reduces false-positive signalling by non-specific surface adsorption. The surface-free nature of the assay allows the assay to be operated in a single step. By only using a single affinity reagent per target, the complexity of assay design is reduced because validated, noncross-reactive affinity probe pairs and multi-epitopic targets are not required.

Table 1 below provides a comparison of the present invention vs ELISA and bead-based immunoassays:

Table 1

| | |
|---|---|
| ELISA and bead-based immunoassays | Present invention |
| Surface-based | Free solution |
| Capture efficiency determined by K_{d} andsurface concentration of affinity probe | Complete binding of target molecules achievable even with low affinity probes |
| Multi-step assay involving washing steps for removal of excess probes and reagents | Single-step assay with fast removal of excess affinity probe |
| Specificity from affinity criterion only | Specificity from combined affinity and electrophoretic criteria |
| Affinity reagent pair | Single affinity reagent |

The device comprises at least one separation region and at least one detection region. In an embodiment, the device comprises a separation region and a detection region. In a further embodiment, the device may comprise more than one separation region and/or more than one detection regions. As such, in an embodiment, the device may comprise a first separation region and a second (third, fourth etc) separation regions. In a further embodiment, the device may comprise a first detection region and a second (third, fourth etc) detection regions. The regions may be configured in series or in parallel depending on requirements. Separation and detection may be grouped (e.g. first separation then second separation then detection) but other configurations are envisaged, for example first separation followed by first detection followed by second separation followed by second detection. The desired configuration is flexible and able to be configured to suit particular needs. Such a system can be used, for example to isolate and carry out a first measurement on a target analyte and then to change the properties of the target analyte and make a second measurement. This could investigate, for example, protein binding, protein folding or measuring other changes to a target biomolecule.

An advantage of the present invention is that it enables non-invasive separation techniques followed by immediate detection and analysis. Target biomolecules can, for example, remain in their natural conformational state in solution. This allows critical probing of target analytes that has hitherto not been possible, or has not been possible to achieve in a rapid test.

Flow rates within the device can be configured to suit the needs of the region. For example, it may be advantageous to have a different flow rate during separation to the flow rate used during detection. In an embodiment, the flow rate during separation is slower than the flow rate during detection. In a further embodiment, the flow rate during separation is faster than the flow rate during detection. Flow rates can be achieved, for example, by configuring the device geometry to enable different channels or different regions of the device to have different flow rates whilst remaining in fluid connection. Device geometry can be optimised to ensure appropriate flow rates are achieved in each area of the device.

In a preferred embodiment, by directly counting the target biomolecules, it is possible to infer concentration without calibration. As such, the present invention provides a calibration free way to determine the concentration of a target analyte.

The present invention provides methods for purely solution phase sample handling. In an embodiment, the present invention provides a solution-phase only method to separate and investigate a target biomolecule from a fluid sample.

The present invention enabling detection at very low concentrations and/or enabling detection of biomolecules (for example but not limited to proteins) in their natural conformation, is suited to methods of detecting biomarker(s) useful in the clinical diagnosis of disease. The present invention is not limited to the type of biomarker or disease. Non-limiting examples include detecting cancer, neurodegenerative diseases, infectious diseases including viral, bacterial or other pathogens, or cardiovascular disease.

The present invention may be suited to detect aggregate-prone proteins such as tau, alpha-synuclein and huntingtin. These proteins mis-fold into smaller oligomers before forming large aggregates or fibrils. The present invention may be used to detect said oligomers and therefore form an early detection strategy for neurodegenerative diseases including Alzheimer’s disease, Parkinson’s disease, and other synucleinopathies including dementia with Lewy bodies (DLB), multiple system atrophy (MSA), and other rare disorders, such as various neuroaxonal dystrophies, Huntington’s disease, Amyotrophic lateral sclerosis (ALS), or Batten disease.

The ability to detect transient species has significant advantages for early stage disease detection. The invention will now be described with reference to the following non-limiting examples.

### Methodology

### A) Microfluidic Device Fabrication

The microfluidic device was designed using AutoCAD software (Autodesk) and printed on acetate transparencies (Micro Lithography Services). The replica mould for fabricating the device was prepared through a single, standard soft-lithography step (Duffy, D. C.; McDonald, J. C.; Schueller, O. J. A.; Whitesides, G. M. Analytical Chemistry 1998, 70,4974-4984) by spinning SU-8 3050 photoresist (MicroChem Corp.) onto a polished silicon wafer to a height of around 25 µm. The UV exposure step was performed with a custom-built LED-based apparatus (Challa, P. K.; Kartanas, T.; Charmet, J.; Knowles, T. P. Biomicrofluidic 2017, 11, 014113) and the precise height of the features were measured to be 28 µm by a profilometer (Dektak, Bruker). The mould was then used to generate a patterned poly(dimethylsiloxane) (PDMS; Dow Corning) slab, which was further bonded to a thin glass coverslip after both of the surfaces had been activated through an oxygen plasma (Diener electronic, 40% power for 15 seconds). Before injecting the solutions into the channels, the chips were exposed to an additional plasma oxidation step (80% power for 500 seconds) which rendered the channel surfaces more hydrophilic (Tan, S. H.; Nguyen, N. T.; Chua, Y. C.; Kang, T. G. Biomicrofluidic 2010, 4, 1-8).

### B) Preparation Of Protein Samples

The complex between the biotinylated and fluorophore-conjugate DNA sequence (5′-Atto488-CGACATCTAACCTAGCTCACTGAC-Biotin-3′; Biomers, SEQ ID NO: 1) and monovalent streptavidin was formed by mixing 25 pM of the monovalent streptavidin sample with 50 pM of biotinylated probe DNA in 10 mM HEPES (pH 7.4) buffer (Sigma) supplemented with 0.05% Tween-20. Prior to its injection to the chip, the mixture was incubated at room temperature for 5 minutes (monovalent streptavidin sample from the Howarth Lab (University of Oxford)).

Recombinant IgE Kappa (clone AbD18705_IgE) was purchased from Bio-Rad and dissolved to a concentration of 40 nM similarly in 10 mM HEPES (pH 7.4) buffer supplemented with 0.05% Tween-20. The protein sample was then incubated with the aptamer probe (5′-Atto488-TGGGGCACGTTTATCCGTCCCTCCTAGTGGCGTGCCCC-3′; Integrated DNA Technologies, SEQ ID NO: 2), 50 pM) for 10 minutes before the experiment at room temperature prior to their injection to the chip.

α-synuclein fibrils were detected using T-SO508 aptamer (Tsukakoshi, K et al, Anal. Chem. 84, 5542-5547 (2012) (5′-Alexa488-TTTTGCCTGTGGTGTTGGGGCGGGTGCG-3′, (SEQ ID NO: 3), HPLC purified; IDT). Prior to its use, the aptamer (100 µM stock in 1X TE buffer) was heated to 70° C. and cooled to room temperature to facilitate correct folding. The α-synuclein fibrils were prepared by incubating α-synuclein monomer as described in Chen, S. W. et al Proc. Natl. Acad. Sci. U. S. A. 112, E1994-E2003 (2015) and Arter, W. E. et al. bioRxiv 2020.03.10.985804 (2020) doi:10.1101/2020.03.10.985804; and sonicated (10 % power, 30 % cycles for 1 min; Sonopuls HD 2070, Bandelin). Following sonication, the fibrils were spun down and re-suspended in 10 mM HEPES (pH 7.4), 0.05 % Tween-20. The aptamer and the fibrils were then mixed by suspending them into 10 mM HEPES (pH 7.4) buffer supplemented with 0.05 % Tween-20 to final concentrations of 10 nM and 80 nM (monomer equivalent), respectively. The mixture was incubated for 10 minutes before its injection to the chip.

### C) Single-Molecule Confocal Optical Setup

The sample was excited using a 488 nm wavelength laser (Cobolt 06-MLD, 200 mW diode laser, Cobolt), which was directed to the back aperture of a 60X-magnification water-immersion objective (CFI Plan Apochromat WI 60x, NA 1.2, Nikon) using a single-mode optical fibre (P3-488PM-FC-1, Thorlabs) and an achromatic fibre collimator (60FC-L-4-M100S-26, Schäfter/Kirchhoff GmbH). The laser intensity at the back aperture of the objective was adjusted to 150 µW. The beam exiting the fibre was reflected by a dichroic mirror (Di03-R488/561, Semrock), directed to the objective and focussed into the microfluidic chip to a diffraction limited confocal spot. The emitted light from the sample was collected through the same objective and dichroic mirror, and passed through a 30 µm pinhole (Thorlabs) to remove any out-of-focus light. The emitted photons were filtered through a band-pass filter (FF01-520/35-25, Semrock) and then passed to an avalanche photodiode (APD, SPCM-14, PerkinElmer Optoelectronics) connected to a TimeHarp260 time-correlated single photon counting unit (PicoQuant).

The dimensions of the confocal volume were determined by performing an FCS experiment on Atto488 carboxylic acid (100 pM; 150 µW laser power). Using its diffusion coefficient of D = 400 µm²s⁻¹, the effective volume of the confocal volume was evaluated to be V_{eff}= 4:2 fL and the kappa factor to be κ = 6:0, yielding an estimate for the dimensions of the confocal volume as z = 3 µm in its height and around w = 0:4 µm in its width. The correlation analysis was done using the SymPhoTime 64 software package (Picoquant).

### D) Experimental Procedure

For experiments performed on the biotin-streptavidin system, the sample and the co-flowing buffer were injected into the device at a flow rate of 70 and 2000 µL h⁻¹, respectively, and the electrolyte solution from each of its inlets at around 300 µL h⁻¹ using 1 mL glass syringes (Hamilton®). For the single molecule detection on IgE and on the α-synuclein fibrils, these injection flow rates were 100, 1200, 400 µL h⁻¹ and 50, 1200, 200 µL h⁻¹, respectively. The PDMS -glass chip was secured to a motorised, programmable microscope stage (Applied Scientific Instrumentation, PZ-2000FT) and once a stable flow in the device had been established, a potential difference across the device was applied using a 500 V bench power supply (Elektro-Automatik EA-PS 9500-06). To facilitate the application of the field, the power supply had its terminals connected to hollow metal dispensing tips (20G, Intertonics) at the electrolyte outlet channels (Saar, K. L. et al Lab on a Chip 2018, 18,162-170). The photon traces were obtained across the cross-section of the device by translocating the microscope stage across its cross-section using a custom-written Python script that simultaneously controlled the stage movement and the data acquisition at a distance of 4 mm downstream from where the electric field was first applied and at the mid-height of the device.

Each of the experiments was performed in a freshly fabricated PDMS device and current readings were recorded simultaneously to ensure that the efficiencies did not vary between the devices and comparisons could be drawn between the deflected distances at which molecules eluted.

### E) Data Analysis

The passing molecules were identified and distinguished from the background by requiring the interphoton arrival times to remain short for the arrival of a number of consecutive photons - this approach has been shown to allow effective distinguishing between background photons that arrive at short intervals by chance and those that originate from a fluorescent dye passing the laser spot and emitting photons (Fries, J. R. et al, The Journal of Physical Chemistry A 1998, 102,6601-6613 and Schaffer, J. et al, The Journal of Physical Chemistry A 1999, 103,331-336). Specifically, an inter-photon time threshold of 100 µs was used for the analysis of the biotin-streptavidin interaction and the IgE sample with consecutive photon arrival events identified as a molecule when a packet of at least 7 photons arrived each with an inter-photon time below threshold. For the analysis of the fibril sample these thresholds were set to 5 µs and 30 photons, respectively. In all cases, before analysis the inter-photon time traces were processed with Lee filter (n = 4) to smoothen regions of constant signal while keeping those with rapid parameter changes, such as the edges of the bursts unaffected.

### F) Simulation of Probe-Target Binding Interaction

Estimations of analyte binding and complex dissociation depicted in FIG. 9b were obtained by examining the binding of probe (P) to its target analyte (A) according to:

$$\text{P} \text{+} \text{A} {⇌}_{{k}_{o f f}}^{{k}_{o n}} \text{PA}$$

where kₒₙ and k_{off} are the rate constants for the formation of the complex and its dissociation, respectively. The simulations in FIG. 9b(i) describe the thermodynamic equilibrium for probes with various dissociation constants K_{d} ranging from 0.1 nM to 1000 nM as a function of its concentration C_{probe}. The simulations in FIG. 9b(ii) illustrate the dissociation of the protein -analyte complex after the removal of the excess reagents relative to the amount of complex that was present at equilibrium. Any changes in the dissociation constant (K_{d} = k_{off}/kₒₙ) were assumed to originate from alterations in the rate constant that govern the dissociation of the complex

$$\left(\text{PA} {\to }^{{k}_{o f f}} \text{P} \text{+} \text{A}\right)$$

rather than changes in the rate constant governing the formation of the complex

$$\left(\text{P} \text{+} \text{A} {\to }^{{}^{{k}_{o n}}} \text{PA}\right)$$

The rate constant for the latter reaction varies between probes (Landry et al, J. Immunol. Methods 417, 86-96 (2015)). Here, we used kₒₙ = 10⁻⁴ M⁻¹S⁻¹, which for a fixed K_{d} yields conservative estimates for k_{off}, and hence, for the rate at which the complex dissociates.

### Example 1 - Microfluidic Device Incorporating Single Molecule Separation and Detection

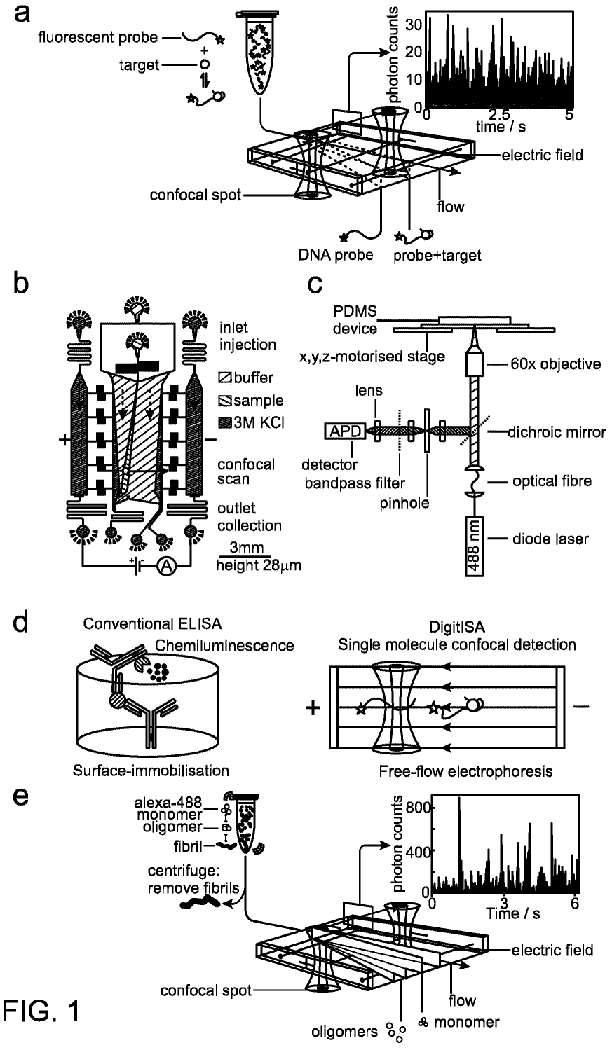

FIG. 1 shows an exemplary device according to an aspect of the present invention. The device incorporates a separation stage and a detection stage. In the specific example give, the device uses microfluidic free-flow electrophoresis (µFFE) combined with downstream high-sensitivity single-molecule fluorescence spectroscopy, enabling direct molecule analysis (e.g. counting) in solution. FIG. 1a shows the principle of the approach.

An exemplary general workflow of the technology is as follows: 1) An analyte solution containing a target biomolecule and additional material is obtained or prepared. 2) Aliquots are withdrawn from the analyte solution and injected into the device as a fluid sample. 3) The fluid sample is subjected to a separation stage. Any suitable separation mechanism is encompassed by the present invention. It can be seen from FIG. 1 that the different biomolecules are separated in a separation region of the device. 4) The separated biomolecules are subjected to a detection step. Any suitable detection means are encompassed by the present invention. The detection is carried out in a detection region of the device. The separation region and the detection region may be separate regions. Alternatively, the separation region and detection region may overlap. For example, it is possible for the biomolecules to be still undergoing separation as they are detected, provided sufficient separation has taken place. 5) The information gathered from the detection step is analysed to determine property or properties of the target biomolecules.

In the example shown in FIG. 1a, in step 3) separation is carried out by means of an electric field. In particular, an electric field is applied perpendicularly to the flow to deflect biomolecules laterally according to their electrophoretic mobility as they flow through the separation channel.

In the example shown in FIG. 1a, in step 4) at constant applied voltage, biomolecules are detected by confocal microscopy. The system can be set to detect biomolecules in various ways, for example continuous scanning or stepwise scanning, collecting data at defined positions. As shown in the example, the confocal volume is scanned through the centre of the device to probe the passage of complexes. This movement is conducted in two modes, by either scanning the confocal spot continuously through the device, or by moving the spot along the same trajectory in a stepwise manner, collecting data at a defined position with each step. In scanning mode, an electropherogram of the system can be rapidly acquired. In stepping mode, bursts of fluorescence corresponding to the passage of single molecules/particles/complexes through the confocal volume over time can be observed as a function of their electrophoretic mobility. The overall trajectory depends on the known device geometry, flow rate, applied voltage and the electrophoretic mobility of the target.

By way of example, for step 5) using a burst-analysis algorithm, the intensities of single-molecule fluorescence events are then identified and analysed (e.g., in resultant histograms of photon counts per event) for downstream analysis.

An exemplary device is shown in more detail in FIG. 1b which shows a micron scale electrophoretic separation unit. The application of electric field allows target biomolecules to be discriminated from non-target biomolecules (in the example shown protein-bound probe molecules can be discriminated from those probe molecules that are not bound to the protein target, owing to a difference in their electrophoretic mobilities).

An exemplary detection region is shown in more detail in FIG. 1c, which shows a single-molecule confocal spectroscopy setup that screens the cross-section of the device and thereby allows the flux of the protein bound probe molecules to be estimated. Other detection techniques are envisaged and encompassed by the present invention.

It can be seen from the example above, that the present invention enables single particle-resolved sensing of target biomolecules through highly controlled, physical separation of molecules in solution on fast timescales.

It will, of course, be apparent that alternative separation techniques and alternative detection techniques can be used with the present invention.

It is also possible to have multiple separation steps and/or multiple detection steps depending on need. This could involve, for example, a first separation step followed by a separate separation step which is either the same or different to the first separation step. It is equally possible to have multiple detection steps so as to gather multiple data points for the target biomolecule, thereby creating a multidimensional picture of the target. It is also possible to combine multiple separation and detection steps, for example having a first separation step, followed by a first detection step, then having a second separation step followed by a second detection step. This and other possibilities are contemplated by the present invention and can be incorporated into the device design.

FIG. 1a shows the principle of resolving heterogeneous protein mixtures. The target biomolecule is complexed with a DNA probe to enable detection but the prior separation step separates both unbound probe and non-target biomolecules such that these unwanted materials do not interfere with the detection step.

FIG. 1e is similar but demonstrates the principle of detection/sensing of biomolecular binding reactions and immuno-complexes. It can be seen that monomers and oligomers are separated during the separation step so that the target analyte can be detected without interference.

The difference between the prior art and present techniques is exemplified in FIG. 1d, which shows conventional ELISAs (and their adaptions) vs the technique of the present invention which allows a direct calibration-free read-out of the biomolecule target in free-solution, in a single step and in a manner where surface-immobilisation of molecules is not required.

The present invention may also include further features, for example, multicolour single-molecule spectroscopy and FRET techniques, other microfluidic separation modalities (for example those described in Arter et al Biophys. Rev. 1-11 (2020) doi:10.1007/s12551-020-00679-4 or Herling et al Lab Chip 18, 999-1016 (2018)), and/or downstream analyses described in Saar et al, Microsystems Nanoeng. 5, 1-10 (2019), the contents or which are hereby incorporated by reference. Such features can further enhance assay parallelization, sensitivity, and/or robustness to experimental noise.

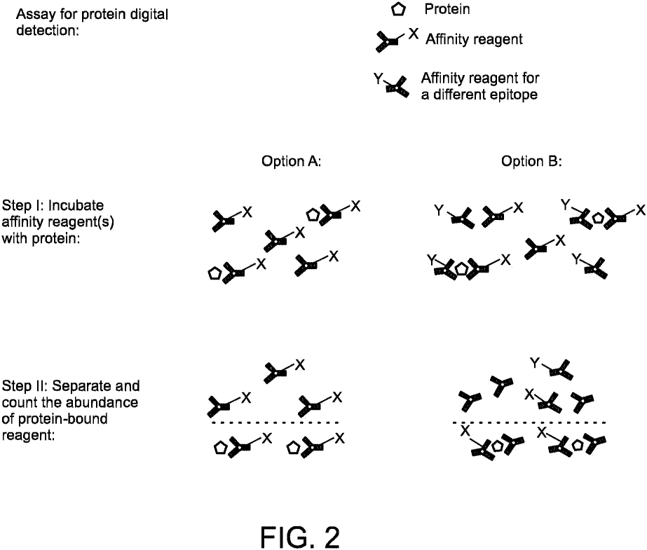

Exemplary separation strategies are shown in more detail in FIG. 2. As has been previously discussed, a critical challenge in the context of biomolecule detection in a complex background is often the requirement to remove molecules, which interfere with the signal amplification or readout. Immobilisation-based approaches achieve this through the use of a washing step on a solid surface. During this step, however, both sample and information might be lost.

The present invention allows for separation of bound and free probe using, for example, a field mediated separation approach. Affinity reagents are used that allow for specific detection of the biomolecule target of interest and bound probe is separated out from the free counterparts based on a difference in their respective physical properties. This is shown in FIG. 2, which shows as Option A in step I incubation of an affinity reagent with a target biomolecule, in this case a protein. By incubating a sample with affinity reagents as e.g. antibodies or aptamers, biomolecule targets of interest can be selectively captured. In step II, biomolecule-bound and non-bound reagents are then separated to quantify the protein concentration.

The present invention makes use of differences in the physical properties of the biomolecule-bound and non-bound affinity reagents. In the present case, the affinity reagents are shown in the same of an antibody but can be any type of affinity reagent, such as an antibody or an aptamer.

Using an affinity reagent can serve as a vehicle to achieve improved or multiplexed detection on-chip, such as a multi wavelength single molecule detection system that can allow detecting multiple targets simultaneously.

It can also serve as a vehicle to perform the detection not molecule-by-molecule but on a “cluster” level by collecting the fraction and amplifying it off-chip, e.g. with PCR, or by absorbing the molecules to a specific location and imaging on a microarray.

If it is desired to increase separation efficiency, and thereby improve the sensitivity of the assay, then a second or further affinity reagent(s) for a different epitope(s) on the target analyte could be included. This is shown in Option B in FIG. 2. Such an approach creates a larger difference in the physical properties of the biomolecule-bound and non-bound form of the probe.

If desired, one or more of the affinity reagent(s) could be labelled, for example fluorescently labelled, depending on the method that is used for downstream detection.

The present invention encompasses using one or more than one affinity reagent(s). Said affinity reagent(s) may optionally be conjugated to a group that may facilitate separation and/or detection. Suitable groups include DNA sequences and fluorophore.

FIG. 2 provides an example where one affinity reagent is conjugated to an X group and a second affinity reagent is conjugated to a Y group. Further affinity reagents are possible, with or without their own conjugated groups. If present, the conjugated groups may be the same or different.

In an embodiment, the present invention uses a first affinity reagent. In an embodiment, the affinity reagent is conjugated to a group X. In a further embodiment, the present invention uses a further affinity reagent. In an embodiment, the further affinity reagent is conjugated to a group Y. Thus, the present invention encompasses using one or more than one affinity reagent and said reagents may or may not be conjugated to groups X, Y and the like. The groups X and Y may be the same or different and may be, for example, a DNA sequence or a fluorophore. It may be possible for X and Y to be different fluorophores.

This approach can open up alternative detection avenues. For instance, groups X and Y may be fluorescent at different wavelengths and co-incidence between the two wavelengths is observed to detect the biomolecular complex. Alternatively, the fluorophores may be chosen so that a FRET even can occur between them and the presence of the biomolecule is detected through FRET. Analogously, when X and Y correspond to DNA sequences, the two sequences can ligate and the complex can be detected such as is done in proximity ligation assays.

Incorporating DNA moieties may allow for enhanced sensitivity in detection of the target biomolecule. In one example, where the affinity reagent comprises a DNA moiety, the separated fraction could be collected as a cluster and the DNA moiety amplified using standard amplification techniques (e.g., PCR). Alternatively, again where the affinity reagent comprises a DNA moiety the separated fraction could be detected on a DNA microarray.

Accordingly, in a further embodiment, the separation step can be carried out before the target biomolecule is complexed with the DNA moiety. Alternatively, the target biomolecule can be complexed with the DNA moiety and then separated. The latter approach is preferred since separation will also separate unbound DNA moieties.

The separation step can be carried out before DNA amplification. Alternatively, separation can be carried out after DNA amplification. Such an approach will result in a much larger target biomolecule to be separated.

Of course, it is also possible that the target biomolecule itself may inherently be separable from other material. In this embodiment, characteristics of the target biomolecule enable sufficient separation to allow for detection of the molecules of interest.

For the avoidance of doubt, the use of affinity reagents (shown as X and Y in FIG. 2) are optional and are not required in embodiments of the present invention.

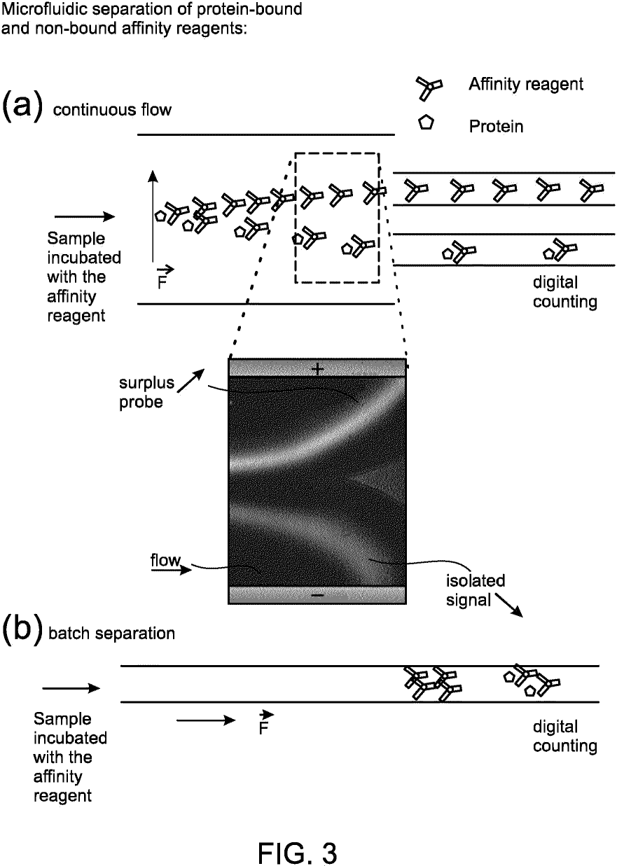

By making use of a field mediated approach (or other suitable separation technique), it is possible to separate the target biomolecule from unbound affinity reagent. This is exemplified in FIG. 3 which shows biomolecule-bound and free probes separated from each other on a microfluidic chip relying on the differences in their physical properties.

It can be seen that once the sample has been incubated with an affinity reagent the fluid sample is subjected to a field to induce separation. In FIG. 3(a), a perpendicular field is applied to the flow causing separation. In the example show, the material flows into separate channels for appropriate detection. It can be envisaged that multiple channels can be configured to capture appropriate biomolecules based on known or predicted deflection.

In FIG. 3(b), a parallel field is shown which causes a batch separation approach. It can be seen that different biomolecules are spaced in the flow channel due to different rates enabling measurements to be taken of the target biomolecules separated from unwanted material. It is noted that this is an example where there may be overlap between the separation and detection regions since the biomolecules may still be undergoing separation as they are detected.

While the field may be electric, any possible field is envisaged by the present invention. For example, suitable fields include magnetic, thermal or solute gradient fields and the like. It should be noted that under a multiple affinity reagent approach shown in FIG. 2 option II, similar separation strategies can be used.

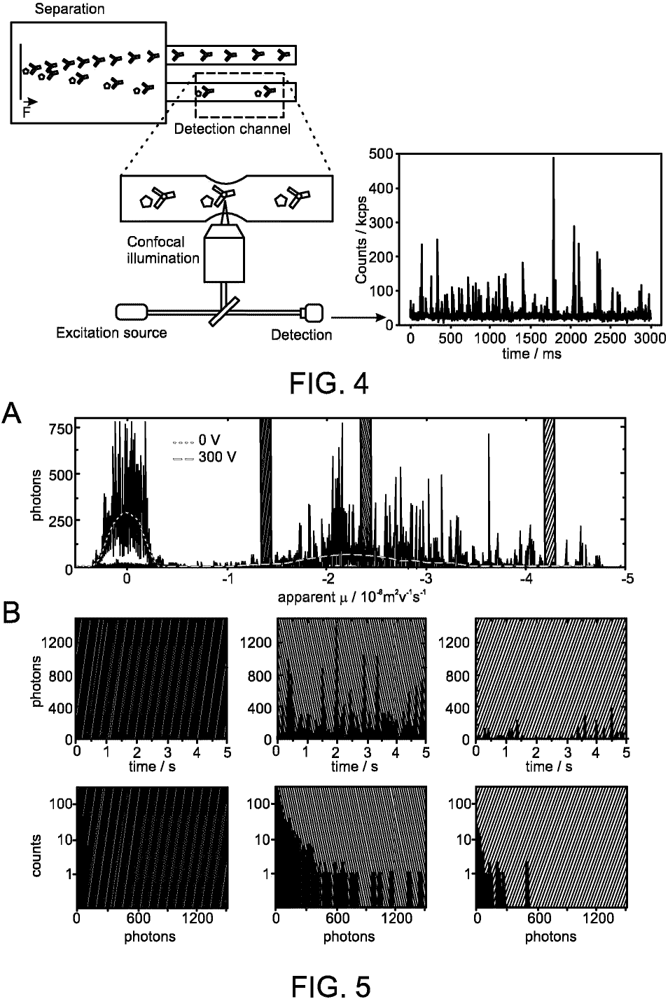

After separation, the biomolecule bound affinity reagents can be detected. An example of a detection region is shown in FIG. 4. In the example shown, single molecule confocal detection is used but the detection can also be achieved by other optical (for example total internal reflection fluorescence (TIRF) microscopy, interferometric scattering (iSCAT) microscopy and the like) or non-optical methods (for example conductance based detection e.g. using nanopores). Moreover, it is possible to functionalise the detection area to capture the molecules and perform imaging based counting or other methodologies.

### Example 2 - Analysis of Multi-Component Aggregation Mixtures

As a first proof-of-concept, a device was prepared to demonstrate the effective analysis of multi-component aggregation mixtures where constituent protein elements are separated according to their differential electrophoretic mobilities.

Specifically, oligomeric α-synuclein (αS) was analysed at the single-complex level, in terms of population heterogeneity and physical properties such as charge and zeta-potential. The ability to characterise αS oligomers is highly important in efforts to unravel the physical mechanisms of neurotoxicity in the progression of Parkinson’s disease since oligomers are considered the major toxic species causing the disease. It is, however, extremely challenging to study using conventional biophysical techniques as oligomers typically exist only transiently as highly heterogeneous mixtures present at low concentrations.

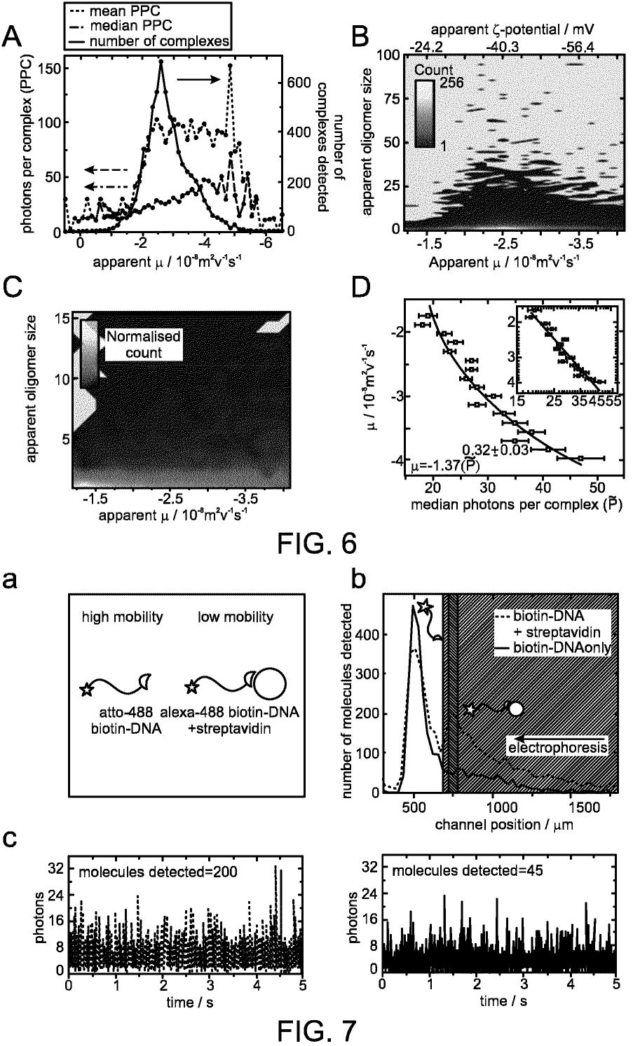

A device comprising an electrophoretic separation region and a single molecule confocal microscopy region was used to detect oligomeric αS at the single-aggregate level. The device demonstrated efficient and high-sensitivity analysis as demonstrated in FIG. 5. FIG. 5a depicts electropherograms generated for αS oligomers formed from Atto-488 labelled monomers in scanning mode. While at 0 V the entire sample was confined to a narrow stream without any means of being able to distinguish between different oligomeric species, at 300 V applied potential, however, efficient electrophoretic separation of different αS oligomeric species was achieved according to their electrophoretic mobility. Stepping mode analysis of the electrophoresed oligomer further revealed differences in burst intensity, dependent upon the step position in the overall electropherogram (FIG. 5b). At lower-mobility positions, single-molecule events of very similar mean burst intensity to that observed for pure αS monomer were observed. At step positions corresponding to higher mobilities, the mean and range of burst intensity per passage event was much higher than that recorded for monomer. Further analysis of the size-mobility relationship revealed that the probability of low-intensity events decreases with increasing mobility, together with an increased probability of high-intensity bursts at high mobility (FIG. 6). Since the median burst intensity describes the variation in oligomer size as a function of mobility, analysis of the scaling coefficient of this trend affords an estimate of size-charge scaling in αS oligomers. Moreover, it is possible to assign an apparent zeta-potential to the observed oligomers on a complex-by-complex basis. This further allows revealing heterogeneity in oligomer surface charge, which may be also be a useful measure with which to probe the properties and behaviours of oligomers. It may also be possible to observe the size of the oligomeric clusters.

It can be seen that the present invention enables the separation of important transient target biomolecules. It can also be seen that since the biomolecules are separated in solution without affecting their conformational structure, critical measurements can be taken in-situ enabling for the first time key information to be gathered in real time.

The example demonstrates single-aggregate complex separation and single particle-resolved sensing. The example demonstrates analysis in a way that has heretofore not been possible and enables unprecedented insights into the physico-chemical nature of multi-component aggregation mixtures.

While the example is shown with the specific αS oligomer, it is universally applicable for sensing applications of heterogeneous protein mixtures.

### Example 3 - Sensing Protein Binding Reactions in a Calibration Free Manner

The sensitive detection of protein complexes is a key objective in many areas of biomolecular science, ranging from biophysics to diagnostics. However, all analytical techniques available to date that enable protein sensing (e.g., ELISA) rely on the paradigm of antibody-antigen reactions to afford specificity for protein sensing. The present invention enables the separation and detection in a simple manner making use of intrinsic physical properties of the biomolecules in solution thereby avoiding surface-mediated immobilisation of analytes.

Avoiding immobilisation also allows the present invention to be suitable for monoepitopic targets.

The present invention also involves a limited number of assay steps increasing the reliability and simplicity of the test thereby making the process scalable and reproducible.

The present invention can also be calibration free when the total amount of analyte is determined through direct digital (single target) counting.

In this example, the assay is demonstrated on the binding of a biotinylated and fluorescently -labelled nucleotide probe strand to monovalent streptavidin as the target. The formation of a biotin-streptavidin complex was evaluated.

A biotinylated and fluorophore-conjugated DNA sequence was mixed with monovalent streptavidin. This is shown graphically in FIG. 7a. This interaction mimics the binding of a protein molecule to its affinity reagent with the binding interaction being very well defined and of a strong affinity. The sample, including 25 pM of the monovalent streptavidin and 50 pM of the biotinylated DNA, was injected into the free-flow electrophoresis separation region (chamber) fabricated in PDMS as per the Methodology section. The sample stream was surrounded by a carrier medium (the sample preparation medium) upon its entry to the separation region, so that it formed a narrow stream at the centre of the chamber. To discriminate the streptavidin bound and non-bound biotin species from each other, an electric potential of 150 V was applied perpendicularly to the direction of flow with a co-flowing electrolyte solution used to ensure the application of stable electric fields as described earlier (see Saar, K. L et al, Lab on a Chip 2018, 18,162-170 and Arter, W. E. et al Analytical chemistry 2018, 90,10302-10310).

To demonstrate that the applied electric field allowed a discrimination between the biotin-bound and non-bound streptavidin molecules, the cross-section of the microfluidic separation chamber was scanned by first injecting the mixture into one microfluidic chip and then injecting a control sample including only the biotinylated DNA with no streptavidin into a different identically fabricated chip. The scanning steps were performed at the mid-height of the channel at a distance of 4 mm downstream from the position where the sample first entered the electric field (FIG. 1). For both devices, the electropherogram was recorded under two conditions - with no field applied across the device and with an electric potential of 150 V applied. While the latter conditions enabled a discriminating the biotin-streptavidin complex the free biotin species, the former conditions ensured that any deviations in the scanning angle between the two devices could be eliminated.

The number of arrived photons at each of the scanned positions was estimated using a combined interphoton time and photon count threshold as described in the Methodology section. This approach has been shown to enable effective distinction between the photons that originate from fluorescent molecules and those that correspond to a background (Fries, J. R. et al, The Journal of Physical Chemistry A 1998, 102, 6601-6613 and Schaffer, J. et al, The Journal of Physical Chemistry A 1999, 103,331-336). Using the obtained counts, electropherograms across the cross-section of the separation chamber were obtained for the two samples (FIG. 7b). From these data, we observed that the binding of the streptavidin molecule decreased the electrophoretic mobility of the DNA-conjugated biotin molecules in comparison to the free DNA-conjugated biotin, with the free biotin eluting at a deflected distance of around 500 µm and the streptavidin-biotin complex at a distance of around 750 µm away from the centre line (red line). Indeed, at the former position, only a minimal elution of fluorescent molecules occurred for the control sample (blue line), indicating that this elution position corresponds to that of the biotin-streptavidin complex. Moreover, it was noted that the recorded fluorescence at the position where the unbound-biotin molecules eluted was higher in the control sample than for the case when the streptavidin target was present. This observation further confirmed the integration of the biotin molecules into the complex.

Having confirmed the capability of the platform to detect the formation of the biotin-streptavidin complex, the potential to estimate its concentration was explored. Indeed, while many protein quantification assays, including conventional ELISAs rely on a calibration curve between the observed signal and the concentration of the analyte molecules, the present invention has the potential to directly count the number of passing molecules and thereby yield an estimate of the protein concentration without a requirement for calibration. First, the number of molecules that eluted between a deflected distance of x=700 µm and x=2200 µm was evaluated (FIG. 7b, region shaded in light grey). 2571 molecules for the biotin-streptavidin mixture were recorded in contrast to only 732 molecules for the control sample. The non-zero count for the control sample is likely to originate from impurities in the biotin sample. The difference between the two obtained counts, 1839 molecules, can be attributed to streptavidin-biotin complex. We note that this count corresponds to the molecular flux in the regions where the single-molecule spectra were recorded. The flux of the streptavidin-biotin complex molecules through the full device, can be estimated from the following relationship:

$$\begin{matrix}{F}_{\text{total}} = \frac{{N}_{\text{scanned}}}{t} ⋅ \frac{H ⋅ {d}_{\text{step}}}{\frac{π}{4} ⋅ z ⋅ w} = \\ \frac{1839 \text{molecules}}{5 \text{s}} ⋅ \frac{28 μ \text{m} ⋅ \text{31} \text{.7} μ \text{m}}{\frac{π}{4} ⋅ 3 ⋅ 0.4 μ {\text{m}}^{2}} = 346 000 \text{molecules} {\text{s}}^{- 1}\end{matrix}$$

where N_{scanned} is the molecule count in the regions that were scanned with the laser, t is the time period over which the time traces were recorded, H is the height to which the separation chamber was fabricated and dₛₜₑₚ is the step size at which the single-molecule timetraces were recorded. The cross-section of the laser spot, specifically, its height, h = 3 µm and width, w = 0.4 µm, were estimated from a fluorescent correlation spectroscopy measurement (Methodology section). As the sample was entering the device at a flow rate of Qₛₐₘₚₗₑ = 70 µLh⁻¹, this molecular flux yielded an estimate of:

$$\begin{matrix}{C}_{\text{complex}} = \frac{{F}_{\text{total}}}{Q ⋅ {N}_{A}} = \\ \frac{346 000 \text{molecules} {\text{s}}^{- 1}}{6.02 ⋅ {10}^{23} \text{molecules} {\text{mol}}^{- 1} ⋅ 70 μ \text{L} {\text{h}}^{- 1}} = 29.5 \text{pM}\end{matrix}$$

for the concentration of the streptavidin-biotin complex in the sample. The binding affinity between the monovalent streptavidin and biotin molecules has previously been estimated to be in the femtomolar range. As such, under the conditions used here (25 pM streptavidin and 50 pM biotin), the majority of the monovalent streptavidin in the mixture is expected to be incorporated into the complex. The experiment estimated slightly smaller than predicted concentration, which is likely to originate from a small amount of the molecules adhering to surfaces, such as the microfluidic channels and the tubing and the needle that were used for injecting the sample.

This example demonstrates the ability to utilise a combined separation/detection strategy, in this case using µFFE for target selection on the basis of differential electrophoretic mobilities of the probe and probe-target complex and conventional affinity-based selection in combination with single-molecule detection to digitally analyse the complex formation.

### Example 4

Having established a platform for highly sensitive detection and quantification of biomolecular complexes, an experiment was carried out to demonstrate its application as a direct digital immunosensor assay using IgE as the target of the assay. IgE is a key component of the human immune system, with a particular relevance in allergic responses. An elevated IgE concentration is a defining characteristic of hyper IgE syndrome and IgE myeloma making accurate quantification of this biomarker an important diagnostic procedure.

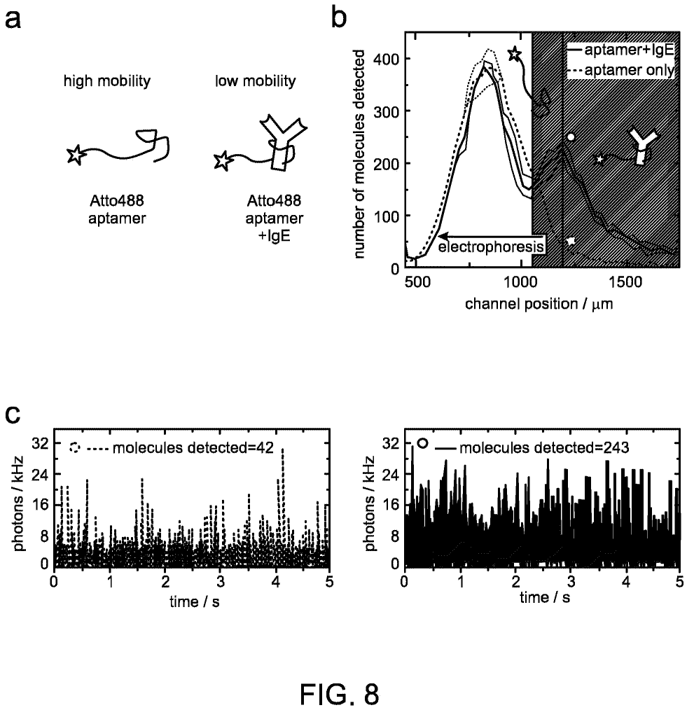

An established IgE aptamer (Wiegand, T. W. et al, The journal of immunology 1996, 157,221-230) labelled with Atto-488 fluorophore was used to detect the presence of IgE molecules. This is shown graphically in FIG. 8a.

IgE (40 nM) and aptamer probe (50 pM) were premixed and injected into the microfluidic separation device and an electropherogram acquired (FIG. 8b, red line) in a similar manner to the streptavidin-biotin system described earlier. As expected, both the free probe and the probe -protein complex were observed in the electropherogram with the complex eluting at a lower deflection value due to its reduced mobility (FIG. 8b, red line). A control experiment with no IgE showed only the free-probe peak with a minimal amount of fluorescence detected at the elution position of the complex (FIG. 8b, blue line). Similarly to before, the photon time traces were used to estimate the flux of fluorescent molecules in the regions where the complex eluted (FIG. 8b, region shaded in light grey). From these data, it was concluded that there were 2920 molecules eluting over a time period of 5 s for the mixture and 1099 molecules for the control sample including only the probe molecules. Using a conversion strategy similar to what was described earlier for the biotin-streptavidin system, this molecular flux value yielded an estimate of 20.5 pM for the concentration of the aptamer-IgE complex.

Using a simple 1:1 binding model for the aptamer-IgE interaction and our knowledge of the concentrations of the IgE bound (c_{complex} = 21.7 pM) and free (c_{free} = 50 pM-21.7 pM = 28.3 pM) forms of the aptamer probe, we calculated the concentration of IgE present in the sample to be c_{IgE} = 38.4 ± 1.6 nM according to c_{IgE}=c_{complex}·(K_{d}+c_{free})/c_{free}. This value showed excellent agreement with the nominal starting concentration of IgE, with the small deviation likely originating from an uncertainty in the reported value of the binding constant K_{d} (Turgeon, R. T. et al Anal. Chem. 82, 3636-3641 (2010) and German, I., et al Anal. Chem. 70, 4540-4545 (1998)). This result demonstrates the efficacy of the present invention in quantitative protein sensing even for relatively weak-binding probes, as enabled by the rapid fractionation of protein bound and unbound probe by the microchip free-flow electrophoresis of the present invention, and provides a route to performing measurements in a regime where the availability of the affinity reagent is limited. Notably, the present invention exploits the fact that detection relies only on a single, monovalent interaction between the probe and the analyte, which allows facile back -calculation of the target concentration from the underlying binary equilibrium, as demonstrated above. Conversely, even for a well-characterized ELISA experiment, such calculation is challenging to realize given the multiple analyte-antibody equilibria that are present in the sandwich-type formats employed in ELISAs.

### Example 5

Having shown the advantages associated with the fast assay timescale, the ability of the assay to directly sense the probe-analyte binding equilibrium by the use of high probe concentration was demonstrated. The binding between α-synuclein fibrils and an aptamer which has been shown previously to weakly bind fibrillar forms of the α-synuclein protein with an approximate K_{d} of 500-1000 nM was investigated (Tsukakoshi, K. et al, Anal. Chem. 84, 5542-5547 (2012)).

Fibrillar α-synuclein is a molecular hallmark of Parkinson’s disease and other synucleinopathies including dementia with Lewy bodies (DLB), multiple system atrophy (MSA), and other rare disorders, such as various neuroaxonal dystrophies. Sensing of α-synuclein aggregates is thus a means for the early detection for these conditions.

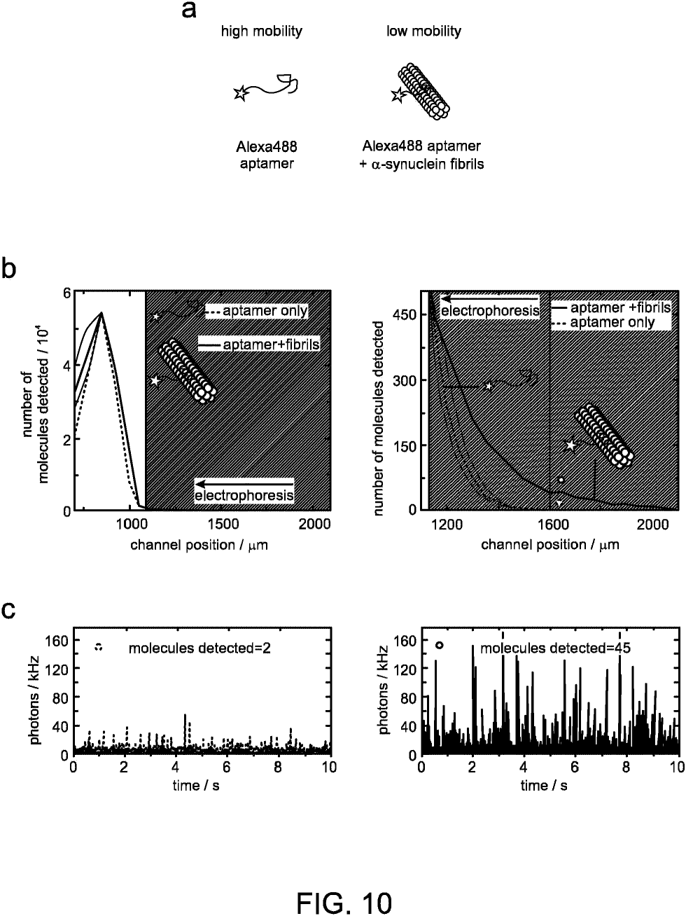

As illustrated in FIG. 10a, binding of the aptamer to the α-synuclein fibrils is proposed to suppress the electrophoretic mobility of the fibrils, thus enabling efficient separation of aptamer -bound fibrils from the unbound probe that is provided in excess.

To demonstrate this capability, a sample of the fibrils with 10 nM of aptamer probe was incubated (see the Methodology section) and acquired an electropherogram across the cross-section of the device while applying a potential difference of 80 V across its terminals (FIG. 10b, blue line). By comparison of the electropherogram for the aptamer-fibril sample relative to the aptamer-only control, fibril-aptamer complexes could be identified at lower electrophoretic mobilities relative to the unbound aptamer peak (FIG. 10b, purple line). There was estimated to be a total of 2066 ± 47 molecules of the unbound aptamer and 926 ± 132 aptamer-fibril complexes eluting in the shaded grey region highlighted in FIG. 10b (1150 µm < x < 2000 µm) over a 10-second timescale (see exemplary time traces in FIG. 10c). Using a similar argument as before, these molecular counts yielded the concentration of protein-bound probe to be 18.7 ± 2.3 pM for the α -synuclein fibrils. This corresponds to a total fibril binding site concentration of 1.0-1.9 nM for a K_{d} range of 500-1000 nM.

It is noted that for a theoretical ELISA experiment with the same analyte concentration and a probe concentration of 1 nM, approximately only 1.9-3.8 pM of fibril would be captured on the surface. Subsequent, rapid dissociation of the probe-aptamer complex due to the weak probe binding strength over the assay timescale would reduce the concentration of bound target well below the assay detection limit. It is clear that the present invention can be used for the detection of weak biomolecular binding interactions, something that is challenging to achieve with conventional multi-step approaches operating over longer time scales.

The example is based on the particular target fibrillar α-synuclein, but it broadly demonstrates the capability of the present invention to be used in the detection and quantification of a wide range of biomolecular and clinically relevant targets. The present invention is an attractive tool for biomolecular analysis and diagnostics as it can, for the first time, reliably detect very low concentrations of target analytes making use of a wide range of probes.

To detect small concentrations of target, it may be desirable to introduce a pre-concentration unit. The modular nature of the devices according to the present invention lends itself well to this strategy.

It is also noted that the sensitivity of the assay is not affected by the dissociation constant of the protein-aptamer pair as the system can be operated with excess levels of probe relative to the target as long as the two populations can be separated.

The sensitivity of the assay could be improved yet further by optimizing the region of data acquisition to maximize the signal-to-background ratio (i.e., 〈nₘᵢₓₜᵤᵣₑ〉/〈n_{control}〉) and minimize s_{control}. For example, altering the acquisition window to between 1600 µm < x < 2100 µm affords a significant reduction in LOD from 6.5 to 0.55 pM.

By combining a physical separation step and single molecule detection into a single device, the present invention creates a simple assay design involving only a single affinity reagent and no washing or blocking steps. This contrasts with incumbent protein detection systems that require a surface-immobilisation step followed by an multistep protocol to remove excess unbound probe molecules (as shown in FIG. 1d).

A physical separation step opens up an additional possibility to use the assay for detecting multiple forms of the same target. For example, the present invention enables a capability to discriminate between monomeric and oligomeric populations of the same protein target. By way of example, these species change the electrophoretic mobility of the probe differently and the corresponding complexes would hence elute at different positions from an electrophoretic separation unit. Such distinction is not possible by previously demonstrated protein detection assays that rely on a combination of a surface-immobilisation and a washing step to remove excess probe.

Generally, by combining single-molecule separation and single-molecule detection into a single device, the present invention enables, for example, the digital detection of biomolecule interaction events. The present invention combines 1) same-phase/homogenous phase, rapid isolation of analyte signal (for example isolating specific proteins (or other biomolecules) from heterogeneous mixtures, or immune-complex from unbound immuno-probe) with simultaneous detection with single-molecule sensitivity. Following this approach allows for purely solution phase sample handling and avoids the need for surfaces or matrix. This enables high sensitivity and the ability to analyse analytes in ways that were not possible before.

The present invention provides a highly sensitive biomolecule detection and quantification strategy. The present invention also provides a direct digital immunoassay. Further, the invention is well suited to monoepitopic targets. In addition to providing a very high level of sensitivity down to sub-picomolar levels, the present invention is implemented in a surface-immobilisation free manner and can thus be used for performing protein detection assays in a single step in contrast to incumbent assays that rely on surface-mobilisation and an array of washing steps. The surface-free nature of the assay enables suppressing non-specific binding events and may thereby provide a fresh route to increasing the sensitivity of the current state-of-the-art biomolecule detection methods, where non-specific electrostatic binding events to surfaces are considered a key contributor to the background signal. As well as increasing sensitivity, new analysis techniques become possible and the present invention therefore provides clear advantages over conventional ELISA techniques.

The present invention can achieve analysis of physical properties, for example the number of fluorophores, net charge and zeta-potential with single-complex or single-molecule resolution. The present invention can achieve sub-dissociation coefficient sensitivity in sensing due to the possibility of using a large excess of probe relative to the target. Single-molecule sensitivity and high throughput of continuous detection (for example via free-flow electrophoresis) means that even sub-pM quantities of bound target can be detected. The present invention enables immuno -sensing possibilities for mono-epitopic targets. The present invention has the capability to distinguish between different physical forms of the same target (for example aggregated vs non-aggregated). The present invention can also infer concentrations without calibration.

Further advantages of the present invention are that no surfaces required as in conventional or bead-based ELISA. Surface-binding of reagents and analytes reduces specificity (non-specific binding to surfaces) and masks the true solution-phase behaviour of sensing or analytical targets (binding sites for proteins inhibited by surfaces). The present invention overcomes these disadvantages. The present invention has the advantage of speed over prior technologies. Fast signal isolation and simultaneous detection on the order of seconds is a significant improvement over ELISA which typically takes hours. The present invention also allows multiple analytes to be observable with the same probe as discussed herein. For example, selective probes can bind to multiple targets in same mixture, with specificity afforded by particular characteristics during the separation phase (for example electrophoretic mobility of probe-target complex). It is also possible to include multiplexing with multiple wavelengths when undergoing detection. Separation techniques can be utilised to obtain size-based information as well as detection information for, for example, oligomeric species. For example, the brightness of detected complexes indicates their size in terms of monomer units. In addition, immuno-sensing is possible with mono-epitopic targets rather than being restricted to biepitopic targets as for sandwich-ELISA.

In conclusion, the present invention provides a surface- and calibration-free platform for the highly sensitive detection and quantification of important target analytes, for example clinically relevant protein targets in solution.

In contrast to incumbent assays that rely on surface-immobilization and serial washing and incubation steps, the present invention operates entirely in solution, does not require washing steps, and performs detection with single-molecule sensitivity in a single step using only a single affinity reagent. The assay format further combines affinity selection with physical separation and thus provides an additional criterion for target detection to afford high specificity and selectivity in the sensing process. The present invention provides a fundamentally new route to surface-free specificity, increased sensitivity, and reduced complexity in state-of-the-art protein detection and biomedical analysis.

Due to in-solution analyte capture and fast removal of excess probe, arbitrarily high concentrations of binding probes can be employed. This enables quantitative analyte-capture regardless of capture probe affinity and further allows the probe-analyte binding interaction to be maintained during the entire sensing process.

This enables relatively weak-binding capture reagents (for example K_{d} = 10- 1000 nM), in contrast to conventional approaches, which require highly-optimized probes with sub-nanomolar affinity such as ultra-high-affinity antibody, whose production is non-trivial and not feasible for some biomarker targets.

This opens up the possibility to use commonly overlooked affinity reagents, such as aptamers, in quantitative single-molecule sensing. Such reagents can be fast and inexpensive to manufacture.

The present invention provides a new paradigm for high sensitivity biomarker (e.g. protein biomarker) sensing and releases constraints of conventional immunosensing approaches in terms of the thermodynamic and kinetics of the immunoprobe-analyte interaction.

The present invention can be used across a wide range of applications. This includes the detection and quantification of a wide range of biomolecular and clinically relevant targets. The present invention is an attractive tool for biomolecular analysis and diagnostics as it can, for the first time, reliably detect very low concentrations of target analytes making use of a wide range of probes.

### Example 6

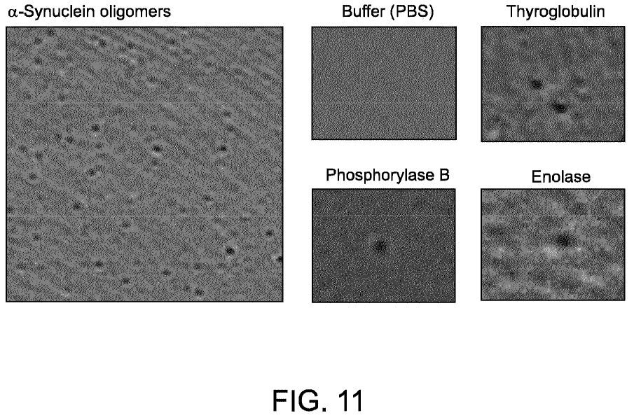

A further experiment was conducted to demonstrate the ability to conduct label-free detection of single protein molecules and protein assemblies using an interferometric scattering (iSCAT) microscope. A linearly polarized continuous-wave laser at 445 nm was used for illumination. The laser beam was passed through acousto-optic deflectors for beam scanning at kHz frequency and then focused by a lens onto the back focal plane of an oil-immersion objective to create flat-field illumination of an area 100 µm². The beam was additionally reflected by a polarizing beam splitter and passed through a quarter-wave plate to separate the excitation from the emission pathway. The scattered and reflected components from the sample and the coverslip were collected by the same objective, passed through by the beam splitter and then imaged with a lens onto a CMOS camera. Protein samples were prepared as stock solutions in phosphate buffered saline (PBS). Thyroglobulin, Phosphorylase B, Enolase, and PBS buffer were from Sigma Aldrich. α-synuclein oligomers were prepared as described in S. W. Chen, et al., Structural characterization of toxic oligomers that are kinetically trapped during α-synuclein fibril formation. Proc. Natl. Acad. Sci. U. S. A. 112, E1994-E2003 (2015). Measurements were performed in polydimethylsiloxane (PDMS) observation chambers on freshly cleaned cover glass slides. To observe single molecule iSCAT landing events, sample chambers were filled with aqueous buffer (PBS), and stock solutions of proteins were added in small aliquots, yielding final protein concentrations of 10-50 nM. To verify that iSCAT binding events originate from single protein molecules or assemblies, first the signal level of buffer solution was monitored and then the protein stock solution added to start detecting single molecule binding events. To generate iSCAT images, images were average over a given number of raw images acquired at 1000 frames per second and subtracted in batches to obtain the differential contrast iSCAT image. The results are shown in FIG. 11.

### References

1. Galasko, D. Biomarkers for Alzheimer’s disease - Clinical needs and application. J.Alzheimer’s Dis. 8, 339-346 (2005).
2. Meriggioli, M. N. Use of immunoassays in neurological diagnosis and research. Neurol. Res. 27, 734-740 (2005).
3. Srinivas, P. R., Kramer, B. S. & Srivastava, S. Trends in biomarker research for cancer detection. Lancet Oncol. 2, 698-704 (2001).
4. Rifai, N., Gillette, M. A. & Carr, S. A. Protein biomarker discovery and validation: the long and uncertain path to clinical utility. Nat. Biotechnol. 24, 971-83 (2006).
5. Kelley, S. O. et al. Advancing the speed, sensitivity and accuracy of biomolecular detection using multi-length-scale engineering. Nat. Nanotechnol. 9, 969-80 (2014).
6. Wild, D. The immunoassay handbook. Theory and Applications of Ligand Binding, ELISA and Related Techniques. (Elsevier Ltd., 2013).
7. Wilson, R. Sensitivity and specificity: twin goals of proteomics assays. Can they be combined? Expert Rev. Proteomics 10, 135-149 (2013).
8. Giljohann, D. A. & Mirkin, C. A. Drivers of biodiagnostic development. Nature 462, 461-464 (2009).
9. Engvall, E. & Perlmann, P. Enzyme-linked immunosorbent assay (ELISA) quantitative assay of immunoglobulin G. Immunochemistry 8, 871-874 (1971).
10. Lequin, R. M. Enzyme immunoassay (EIA)/enzyme-linked immunosorbent assay (ELISA). Clin. Chem. 51, 2415-2418 (2005).
11. Sano, T., Smith, C. L. & Cantor, C. R. Immuno-PCR: very sensitive antigen detection by means of specific antibody-DNA conjugates. Science 258, 120-2 (1992).
12. De La Rica, R. & Stevens, M. M. Plasmonic ELISA for the ultrasensitive detection of disease biomarkers with the naked eye. Nat. Nanotechnol. 7, 821-824 (2012).
13. Stern, E. et al. A nanoelectronic enzyme-linked immunosorbent assay for detection of proteins in physiological solutions. Small 6, 232-8 (2010).
14. Shim, J. et al. Ultrarapid Generation of Femtoliter Microfluidic Droplets for Single-Molecule-Counting Immunoassays. ACS Nano 7, 5955-5964 (2013).
15. Rissin, D. M. et al. Single-molecule enzyme-linked immunosorbent assay detects serum proteins at subfemtomolar concentrations. Nat. Biotechnol. 28, 595-599 (2010).
16. Geiss, G. K. et al. Direct multiplexed measurement of gene expression with color-coded probe pairs. Nat. Biotechnol. 26, 317-325 (2008).
17. Gold, L. et al. Aptamer-Based Multiplexed Proteomic Technology for Biomarker Discovery. PLoS One 5, e15004 (2010).
18. Chang, L. et al. Single molecule enzyme-linked immunosorbent assays: Theoretical considerations. J. Immunol. Methods 378, 102-115 (2012).
19. Sormanni, P., Aprile, F. A. & Vendruscolo, M. Third generation antibody discovery methods: in silico rational design. Chem. Soc. Rev. 47, 9137-9157 (2018).
20. Knowles, T. P. J., Vendruscolo, M. & Dobson, C. M. The amyloid state and its association with protein misfolding diseases. Nat. Rev. Mol. Cell Biol. 15, 384-396 (2014).
21. Arter, W. E. et al. Combining Affinity Selection and Specific Ion Mobility for Microchip Protein Sensing. Anal. Chem. 90, 10302-10310 (2018).
22. Saar, K. L. et al. On-chip label-free protein analysis with downstream electrodes for direct removal of electrolysis products. Lab Chip 18, 162-170 (2018).
23. Saar, K. L., Muller, T., Charmet, J., Challa, P. K. & Knowles, T. P. J. Enhancing the Resolution of Micro Free Flow Electrophoresis through Spatially Controlled Sample Injection. Anal. Chem. 90, 8998-9005 (2018).
24. Howarth, M. et al. A monovalent streptavidin with a single femtomolar biotin binding site. Nat. Methods 3, 267-273 (2006).
25. Fries, J. R., Brand, L., Eggeling, C., Köllner, M. & Seidel, C. A. M. Quantitative identification of different single molecules by selective time-resolved confocal fluorescence spectroscopy. J. Phys. Chem. A 102, 6601-6613 (1998).
26. Schaffer, J. et al. Identification of Single Molecules in Aqueous Solution by Time-Resolved Fluorescence Anisotropy. J. Phys. Chem. A 103, 331-336 (1999).
27. Grimbacher, B., Holland, S. M. & Puck, J. M. Hyper-IgE syndromes. Immunol. Rev. 203, 244-50 (2005).
28. Ogawa, M., Kochwa, S., Smith, C., Ishizaka, K. & McIntyre, O. R. Clinical aspects of IgE myeloma. N. Engl. J. Med. 281, 1217-1220 (1969).
29. Wiegand, T. W. et al. High-affinity oligonucleotide ligands to human IgE inhibit binding to Fc epsilon receptor I. J. Immunol. 157, 221-30 (1996).
30. Tuerk, C. & Gold, L. Systematic evolution of ligands by exponential enrichment: RNA ligands to bacteriophage T4 DNA polymerase. Science 249, 505-10 (1990).
31. Ellington, A. D. & Szostak, J. W. In vitro selection of RNA molecules that bind specific ligands. Nature 346, 818-822 (1990).
32. Zhuo, Z. et al. Recent Advances in SELEX Technology and Aptamer Applications in Biomedicine. Int. J. Mol. Sci. 18, 2142 (2017).
33. Lollo, B., Steele, F. & Gold, L. Beyond antibodies: New affinity reagents to unlock the proteome. Proteomics 14, 638-644 (2014).
34. Turgeon, R. T., Fonslow, B. R., Jing, M. & Bowser, M. T. Measuring aptamer equilibria using gradient micro free flow electrophoresis. Anal. Chem. 82, 3636-3641 (2010).
35. German, I., Buchanan, D. D. & Kennedy, R. T. Aptamers as ligands in affinity probe capillary electrophoresis. Anal. Chem. 70, 4540-4545 (1998).
36. Campbell, J., Pollock, N. R., Sharon, A. & Sauer-Budge, A. F. Development of an automated on-chip bead-based ELISA platform. Anal. Methods 7, 8472-8477 (2015).
37. Tsukakoshi, K., Abe, K., Sode, K. & Ikebukuro, K. Selection of DNA aptamers that recognize α-synuclein oligomers using a competitive screening method. Anal. Chem. 84, 5542-5547 (2012).
38. Parnetti, L. et al. CSF and blood biomarkers for Parkinson’s disease. The Lancet Neurology 18, 573-586 (2019).
39. Streets, A. M. & Huang, Y. Microfluidics for biological measurements with single-molecule resolution. Curr. Opin. Biotechnol. 25, 69-77 (2014).
40. Kim, S. et al. High-throughput single-molecule optofluidic analysis. Nat. Methods 8, 242-5 (2011).
41. Wunderlich, B. et al. Microfluidic mixer designed for performing single-molecule kinetics with confocal detection on timescales from milliseconds to minutes. Nat. Protoc. 8, 1459-1474 (2013).
42. Arter, W. E., Levin, A., Krainer, G. & Knowles, T. P. J. Microfluidic approaches for the analysis of protein-protein interactions in solution. Biophys. Rev. 1-11 (2020). doi:10.1007/s12551-020-00679-4
43. Herling, T. W., Levin, A., Saar, K. L., Dobson, C. M. & Knowles, T. P. J. Microfluidic approaches for probing amyloid assembly and behaviour. Lab Chip 18, 999-1016 (2018).
44. Saar, K. L. et al. Rapid two-dimensional characterisation of proteins in solution. Microsystems Nanoeng. 5, 1-10 (2019).
45. Duffy, D. C., McDonald, J. C., Schueller, O. J. A. & Whitesides, G. M. Rapid prototyping of microfluidic systems in poly(dimethylsiloxane). Anal. Chem. 70, 4974- 4984 (1998).
46. Arter, W. E. et al. Digital Sensing and Molecular Computation by an Enzyme-Free DNA Circuit. ACS Nano (2020). doi:10.1021/acsnano.0c00628
47. Challa, P. K., Kartanas, T., Charmet, J. & Knowles, T. P. J. Microfluidic devices fabricated using fast wafer-scale LED-lithography patterning. Biomicrofluidics 11, 014113 (2017).
48. Tan, S. H., Nguyen, N.-T., Chua, Y. C. & Kang, T. G. Oxygen plasma treatment for reducing hydrophobicity of a sealed polydimethylsiloxane microchannel. Biomicrofluidics 4, 032204 (2010).
49. Chen, S. W. et al. Structural characterization of toxic oligomers that are kinetically trapped during α-synuclein fibril formation. Proc. Natl. Acad. Sci. U. S. A. 112, E1994-E2003 (2015).
50. Arter, W. E. et al. Rapid Fractionation and Characterisation of Alpha-Synuclein Oligomers in Solution. bioRxiv 2020.03.10.985804 (2020). doi:10.1101/2020.03.10.985804
51. Krainer, G., Hartmann, A. & Schlierf, M. FarFRET: Extending the Range in Single-Molecule FRET Experiments beyond 10 nm. Nano Lett. 15, (2015).
52. Krainer, G. et al. A minimal helical-hairpin motif provides molecular-level insights into misfolding and pharmacological rescue of CFTR. Commun. Biol. 1, 154 (2018).
53. Enderlein, J., Robbins, D. L., Ambrose, W. P. & Keller, R. A. Molecular Shot Noise, Burst Size Distribution, and Single-Molecule Detection in Fluid Flow: Effects of Multiple Occupancy. J. Phys. Chem. A 102, 6089-6094 (1998).
54. Landry, J. P., Ke, Y., Yu, G. L. & Zhu, X. D. Measuring affinity constants of 1450 monoclonal antibodies to peptide targets with a microarray-based label-free assay platform. J. Immunol. Methods 417, 86-96 (2015).
55. Draper, M. C. Integrating Microcapillary Electrophoresis and Droplet Microfluidics for Sequential On-Chip Multi-Process Chemical Analysis. PhD Thesis (2014).
56. Li, Q., Zhu, Y., Zhang, N.-Q., Fang, Q. Automatic Combination of Microfluidic Nanoliter-Scale Droplet Array with High-Speed Capillary Electrophoresis. Scienfitic Reports, 6:26554, 1-9 (2016).
57. Pan, Y., Sackmann, E. K., Wypisniak, K., Hornsby, M., Datwani, S. S., Herr, A. E. Determination of equilibrium dissociation constants for recombinant antibodies by high-throughput affinity electrophoresis. Scientific Reports, 6:39774, 1-11 (2016).
58. Ouimet, C. M., D’Amico, C. L., Kennedy, R. T. Droplet sample introduction to microchip gel and zone electrophoresis for rapid analysis of protein-protein complexes and enzymatic reactions. Analytical and Bioanalytical Chemistry, 611, 6155-6163 (2019).
59. Kuswandi, B., Nuriman, N., Huskens, J., Verboom, W. Optical sensing systems for microfluidic devices: A review. Analytica Chimica Acta, 601, 141-155 (2007).
60. Yates, E. V., Muller, T., Rajah, L., De Genst, E. J., Arosio, P., Linse, S., Vendruscolo, M., Dobson, C. M., Knowles, T. P. J. Latent analysis of unmodified biomolecules and their complexes in solution. Nat. Chem., 7, 802-809 (2015).
61. WO 2019/126388
62. US 2005/133370
63. S. W. Chen, et al., Structural characterization of toxic oligomers that are kinetically trapped during α-synuclein fibril formation. Proc. Natl. Acad. Sci. U. S. A. 112, E1994-E2003 (2015)

The invention will now be defined by one or more of the following clauses:
1. A method of investigating a target biomolecule comprising:
   a) adding a suitable affinity reagent to a solution comprising a target biomolecule to form a fluid sample comprising bound affinity reagent-target biomolecule and unbound affinity reagent;
   b) introducing said fluid sample into a microfluidic device comprising a separation region and a detection region configured for highly sensitive, such as single molecule counting or digital, detection of said target biomolecule;
   c) separating said bound affinity reagent-target biomolecule from unbound affinity reagent target biomolecule in said separation region; and
   d) performing highly sensitive, such as single molecule counting or digital, detection on said bound affinity reagent-target biomolecule in said detection region.
2. The method of clause 1 wherein steps a) and b) are reversed such that the fluid sample is introduced into the microfluidic device before the affinity reagent is added.
3. A method of investigating a target biomolecule comprising:
   a) introducing a fluid sample comprising a heterogeneous mixture of material including a target biomolecule into a microfluidic device comprising a separation region and a detection region configured for highly sensitive, such as single molecule counting or digital, detection of said target biomolecule;
   b) separating said target biomolecule from said heterogeneous mixture in said separation region; and
   c) performing highly sensitive, such as single molecule counting or digital detection on said target biomolecule in said detection region.
4. The method according to any preceding clause, wherein the method analyses multiple target analytes.
5. The method according to any preceding clause, wherein the separation step is carried out in a continuous free flow or non-continuous batch process.
6. The method according to any preceding clause, wherein the detection step is carried out using continuous scanning or stepwise scanning.
7. The method according to any preceding clause, wherein the method uses digital counting of target biomolecules.
8. The method according to any preceding clause, wherein the method takes less than 2 h from introducing the fluid sample to detecting the separated target biomolecule, preferably less than 1 h, less than 10 minutes, less than 1 minute, less than 30 seconds or less than 10 seconds.
9. The method according to any preceding clause, wherein a high concentration of affinity reagent is used, particularly an excess of affinity reagent as compared to the target biomolecule.
10. The method according to clause 9, wherein the affinity reagent concentration is greater than 2 nM, greater than 5 nM, greater than 10 nM, greater than 50 nM, greater than 100 nM, greater than 200 nM, greater than 500 nM, greater than 1 µM, greater than 10 nM, greater than 1 µM, greater than 10 µM, greater than 100 µM, greater than 500 µM, greater than 1 mM, greater than 10 mM, greater than 100 mM, or greater than 500 mM.
11. The method according to any preceding clause, wherein the affinity reagent has a dissociation constant K_{d} of 1 nM or more, 5 nM or more, 10 nM or more, 100 nM or more, or 1000 nM or more.
12. The method according to any preceding clause, wherein the affinity reagent is selected from nucleic acids, polypeptides and peptides and fragments thereof, ribonucleoproteins, a protein-nucleic acid complex, antibodies, antibody fragments, antigen binding antibody fragments, nanoparticles, nanobodies, viruses or viral-like particles, enzymes, aptamers, affimers and other non-antibody binding proteins/molecules; preferably aptamers.
13. The method according to any preceding clause, wherein the separation step takes less than 5 minutes, less than 1 minute, less than 30 seconds, less than 15 seconds, less than 10 seconds, less than 5 seconds or on the order of 1-2 seconds.
14. The method according to any preceding clause, wherein the detection step takes less than 10 minutes, less than 5 minutes, less than 1 minute, less than 30 seconds, less than 15 seconds, less than 10 seconds, less than 5 seconds or on the order of 1-2 seconds.
15. The method according to any preceding clause, wherein the method achieves substantially quantitative target biomolecule (antigen) binding.
16. The method according to any preceding clause, wherein the affinity reagent dissociation constant K_{d} and affinity reagent concentration are selected to ensure target biomolecule-affinity reagent binding during detection, particularly to enable substantially quantitative target biomolecule (antigen) binding and detection.
17. The method according to any preceding clause, wherein a single affinity reagent is used.
18. The method according to any preceding clause, wherein the method does not involve a washing step.
19. The method according to any preceding clause, wherein the method is performed in a single step.
20. The method according to any preceding clause, wherein the affinity reagent-target biomolecule binding interaction is maintained during substantially the entire sensing process.
21. The method according to any preceding clause, wherein the method can detect oligomers.
22. The method according to clause 21, wherein the method can detect oligomers of aggregate-prone proteins including tau, alpha-synuclein and huntingtin.
23. The method according to any preceding clause, wherein the highly sensitive detection is single-molecule detection.
24. The method according to any preceding clause, wherein the microfluidic device comprises:
   a) a separation region configured to separate said target biomolecule from a fluid sample comprising a heterogeneous mixture of material; and
   b) a detection region configured for single-molecule detection of said target biomolecule.
25. The method according to any preceding clause, wherein the target biomolecule is selected from proteins, peptides, modified peptides (including post-translational and chemical labelling modifications), amino acid conjugates of non-proteinaceous nature, non-biological amino acid containing proteins and peptides or amino acid conjugates.
26. The method according to any preceding clause, wherein the target biomolecule is monoepitopic.
27. The method according to any preceding clause, wherein the target biomolecule is a transient species.
28. The method according to any preceding clause, wherein the device is configured to investigate a plurality target biomolecules.
29. The method according to any preceding clause, wherein the fluid sample comprises a solvent system, one or more target biomolecules and one or more non-target materials.
30. The method according to any preceding clause, wherein the solvent system is an aqueous solvent system, or wherein the solvent system is a buffered solvent system, including phosphate buffer, phosphate-buffered saline, tris-HCl, HEPES, acetate buffer, and borate buffer.
31. The method according to any preceding clause, wherein the microfluidic device is manufactured by lithography, injection moulding or 3D printing, preferably lithography.
32. The method according to any preceding clause, wherein the separation region comprises a region of the microfluidic device which causes the target biomolecule to separate using a technique selected from free-flow electrophoresis, capillary electrophoresis, diffusion based separation, isoelectric separation, chemical separation, or sizing based separation; preferably free-flow electrophoresis.
33. The method according to any preceding clause, wherein the separation region resolves heterogenous protein mixtures, resolve immuno-complexes from unbound immuno-probes, or resolves otherwise similar biomolecules which differ in respect of physical properties, for example aggregated vs non-aggregated proteins.
34. The method according to any preceding clause, wherein the detection region comprises a region of the microfluidic device which determines one or more properties of the target biomolecule, wherein the detection technique may be selected from fluorescence spectroscopy, for example confocal microscopy, including single wavelength or multi-wavelength confocal microscopy; scattering-based readouts, such as interferometric light scattering; or electrical readouts, such as those obtained in nanopores.
35. The method according to any preceding clause, wherein the detection region counts target biomolecules, and/or wherein the detection region measures one or more properties of the target biomolecule, including size, hydrodynamic radius, molecular weight (M_{w}) charge/ ion binding capacity, iso-electric point (pI), solubility, dipole moment and/or hydrophobicity.
36. The method according to any preceding clause, wherein the target biomolecule is complexed with an affinity reagent; including nucleic acids, oligonucleotides, polypeptides and peptides and fragments thereof, ribonucleoproteins, a protein-nucleic acid complex, antibodies, antibody fragments, antigen binding antibody fragments, nanoparticles, nanobodies, viruses or viral-like particles, enzymes, aptamers, affimers and other non-antibody binding proteins/molecules.
37. The method according to any preceding clause, wherein a further affinity reagent or reagents is/are used, such that two or more affinity reagents are used; said affinity reagents being the same or different but targeting a different epitope.
38. The method according to any preceding clause, wherein one or more of the affinity reagents is/are further linked to a nucleotide moiety; preferably, an oligonucleotide, such as an DNA oligonucleotide or RNA oligonucleotide, or an DNA-aptamer or RNA-aptamer; optionally wherein two or more nucleotide moieties are used, thereby enabling ligation.
39. The method according to any preceding clause, wherein one or more of the affinity reagents is/are further linked to a flurophore moiety; optionally wherein two or more flurophores are used enabling, for example, detection of co-incident light at two wavelengths or recording FRET events.
40. The method according to any preceding clause, wherein the device comprises a plurality of separation regions; and/or a plurality of detection regions.
41. The method according to any preceding clause, wherein the flow rate within the device is configured to assist separation and/or detection; including where flow rate in the separation region is slower than the flow rate in the detection region, or where the flow rate in the separation region is faster than the flow rate in the detection region.
42. The method according to any preceding clause, wherein the separation region and detection region are spatially separated, or wherein the separation region and detection region overlap.
43. The method according to any preceding clause, wherein the device determines target biomolecule concentration without requiring calibration.
44. The method according to any preceding clause, wherein the device comprises modular sub-elements.
45. A method of detecting multiple biomolecules simultaneously comprising:
   a) incubating one or more affinity reagents with a target biomolecule to form target biomolecule bound affinity reagent(s);
   b) separating said target biomolecule bound affinity reagent(s) from unbound affinity reagent and optionally other material; and
   c) detecting one or more properties of said target biomolecule bound affinity reagent(s).
46. The method according to clause 45 wherein, after separation, the target biomolecule bound affinity reagent(s) are detected on a microarray.
47. A microfluidic device for investigating a target biomolecule comprising:
   a) a separation region configured to separate said target biomolecule from a fluid sample comprising a heterogeneous mixture of material; and
   b) a detection region configured for single-molecule detection of said target biomolecule.
48. The device according to clause 47, wherein the target biomolecule is selected from proteins, peptides, modified peptides (including post-translational and chemical labelling modifications), amino acid conjugates of non-proteinaceous nature, non-biological amino acid containing proteins and peptides or amino acid conjugates.
49. The device according to clause 47 or clause 48, wherein the target biomolecule is monoepitopic.
50. The device according to any one of clauses 47 to 49, wherein the target biomolecule is a transient species.
51. The device according to any one of clauses 47 to 50, wherein the device is configured to investigate a plurality target biomolecules.
52. The device according to any one of clauses 47 to 51, wherein the fluid sample comprises a solvent system, one or more target biomolecules and one or more non-target materials.
53. The device according to clause 52, wherein the solvent system is an aqueous solvent system, or wherein the solvent system is a buffered solvent system, including phosphate buffer, phosphate-buffered saline, tris-HCl, HEPES, acetate buffer, and borate buffer.
54. The device according to any one of clauses 47 to 53, wherein the microfluidic device is manufactured by lithography, injection moulding or 3D printing, preferably lithography.
55. The device according to any one of clauses 47 to 54, wherein the separation region comprises a region of the microfluidic device which causes the target biomolecule to separate using a technique selected from free-flow electrophoresis, capillary electrophoresis, diffusion based separation, isoelectric separation, chemical separation, or sizing based separation; preferably free-flow electrophoresis.
56. The device according to any one of clauses 47 to 55, wherein the separation region resolves heterogenous protein mixtures, resolve immuno-complexes from unbound immuno-probes, or resolves otherwise similar biomolecules which differ in respect of physical properties, for example aggregated vs non-aggregated proteins.
57. The device according to any one of clauses 47 to 56, wherein the detection region comprises a region of the microfluidic device which determines one or more properties of the target biomolecule, wherein the detection technique may be selected from fluorescence spectroscopy, for example confocal microscopy, including single wavelength or multi-wavelength confocal microscopy; scattering-based readouts, such as interferometric light scattering; or electrical readouts, such as those obtained in nanopores.
58. The device according to any one of clauses 47 to 57, wherein the detection region counts target biomolecules, and/or wherein the detection region measures one or more properties of the target biomolecule, including size, hydrodynamic radius, molecular weight (M_{w}) charge/ ion binding capacity, iso-electric point (pI), solubility, dipole moment and/or hydrophobicity.
59. The device according to any one of clauses 47 to 58, wherein the target biomolecule is complexed with an affinity reagent; including nucleic acids, oligonucleotides, polypeptides and peptides and fragments thereof, ribonucleoproteins, a protein-nucleic acid complex, antibodies, antibody fragments, antigen binding antibody fragments, nanoparticles, nanobodies, viruses or viral-like particles, enzymes, aptamers, affimers and other non-antibody binding proteins/molecules.
60. The device according to clause 59, wherein a further affinity reagent or reagents is/are used, such that two or more affinity reagents are used; said affinity reagents being the same or different but targeting a different epitope.
61. The device according to clause 59 or clause 60, wherein one or more of the affinity reagents is/are further linked to a nucleotide moiety; preferably, an oligonucleotide, such as an DNA oligonucleotide or RNA oligonucleotide, or an DNA-aptamer or RNA-aptamer; optionally wherein two or more nucleotide moieties are used, thereby enabling ligation.
62. The device according to any one of clauses 59 to 61, wherein one or more of the affinity reagents is/are further linked to a flurophore moiety; optionally wherein two or more flurophores are used enabling, for example, detection of co-incident light at two wavelengths or recording FRET events.
63. The device according to any one of clauses 47 to 62, comprising a plurality of separation regions; and/or a plurality of detection regions.
64. The device according to any one of clauses 47 to 63, wherein the flow rate within the device is configured to assist separation and/or detection; including where flow rate in the separation region is slower than the flow rate in the detection region, or where the flow rate in the separation region is faster than the flow rate in the detection region.
65. The device according to any one of clauses 47 to 64, wherein the separation region and detection region are spatially separated, or wherein the separation region and detection region overlap.
66. The device according to any one of clauses 47 to 65, wherein the device determines target biomolecule concentration without requiring calibration.
67. The device according to any one of clauses 47 to 66, wherein the device comprises modular sub-elements.
68. A microfluidic device as defined in any one of clauses 47 to 67, for use in a method of detecting a biomarker useful in the clinical diagnosis of a disease.
69. The microfluidic device according to clause 68, wherein the disease is cancer, neurodegenerative diseases, infectious diseases including viral, bacterial or other pathogens, or cardiovascular disease.
70. The microfluidic device according to clause 68 or clause 69, wherein the method detects oligomers of aggregate-prone proteins including tau, alpha-synuclein and huntingtin.
71. A method for detecting a target biomolecule biomarker, performed on a sample from a subject, such as a blood sample, tumour sample, tissue sample including brain tissue sample or other sample, said method comprising:
   a) optionally carrying out processing step(s) on said subject sample to obtain a fluid sample;
   b) adding a suitable affinity reagent to said fluid sample to form a fluid sample comprising bound affinity reagent-target biomolecule and unbound affinity reagent;
   c) introducing said fluid sample into a microfluidic device comprising a separation region and a detection region configured for highly sensitive, such as single molecule counting or digital, detection of said target biomolecule;
   d) separating said bound affinity reagent-target biomolecule from unbound affinity reagent target biomolecule in said separation region;
   e) detecting said bound affinity reagent-target biomolecule in said detection region; such that detection of said target biomolecule according to clinically relevant parameters indicates presence of disease.
72. An in vitro method for identifying an individual having risk of disease, including but not limited to cancer, neurodegenerative diseases, infectious diseases including viral, bacterial or other pathogens, or cardiovascular disease, comprising:
   a) optionally carrying out processing step(s) on a sample from a subject sample to obtain a fluid sample;
   b) adding a suitable affinity reagent to said fluid sample to form a fluid sample comprising bound affinity reagent-target biomolecule and unbound affinity reagent;
   c) introducing said fluid sample into a microfluidic device comprising a separation region and a detection region configured for highly sensitive, such as single molecule counting or digital, detection of said target biomolecule;
   d) separating said bound affinity reagent-target biomolecule from unbound affinity reagent target biomolecule in said separation region;
   e) detecting said bound affinity reagent-target biomolecule in said detection region; to detect an individual having risk of said disease.
73. The method according to clause 71 or clause 72, wherein the microfluidic device is as defined in any one of clauses 47 to 67.
74. The method according to any one of clauses 71 to 73, wherein the method is as defined in any one of clauses 1 to 46.
75. The method according to any one of clauses 71 to 74, wherein steps b) and c) are reversed such that the fluid sample is introduced into the microfluidic device before the affinity reagent is added.
76. A method for diagnosing a disease (including but not limited to detecting cancer, neurodegenerative diseases, viral, bacterial or other pathogens, or cardiovascular disease) or an increased risk of having said disease in a subject, comprising the step of using a device according to any one of clauses 47 to 67, or a method according to any one of clauses 1 to 46, to measure a biomarker in a biological sample isolated from said subject.

1. A microfluidic device for investigating a target biomolecule comprising:
a) a separation region configured to separate said target biomolecule from a fluid sample comprising a heterogeneous mixture of material based on a separation technique; and
b) a detection region configured for single molecule detection of said target biomolecule using a single molecule detector;
wherein the target biomolecule is complexed with a probe; wherein the separation region resolves the target biomolecule-probe complex from the unbound probe, and
wherein the detection region is configured to count target biomolecules, and wherein the biomolecules are under flow during detection.

2-6. (canceled)

7. The device according to claim 1, wherein the single molecule may be a single-complex or a single-molecule.

8. The device according to claim 7, wherein the single-complex may be the natural conformational state of the biomolecule .

9. The device according to claim 1, wherein the target biomolecule is selected from proteins, peptides, modified peptides , amino acid conjugates of non-proteinaceous nature, nonbiological amino acid containing proteins and peptides or amino acid conjugates.

10. (canceled)

11. The device according to claim 1, wherein the target biomolecule is a transient species.

12. (canceled)

13. The device according to claim 1, wherein the fluid sample comprises a solvent system, one or more target biomolecules and one or more non-target materials; wherein the solvent system is an aqueous solvent system, or wherein the solvent system is a buffered solvent system, including phosphate buffer, phosphate-buffered saline, tris-HCl, HEPES, acetate buffer, and borate buffer.

14-15. (canceled)

16. The device according to claim 1, wherein the separation region comprises a region of the microfluidic device which causes the target biomolecule to separate using a technique selected from electrophoresis, including but not limited to, free-flow electrophoresis or capillary electrophoresis; diffusion based separation; isoelectric separation; chemical separation, or sizing based separation.

17. (canceled)

18. The device according to claim 1, wherein the detection region comprises a region of the microfluidic device which determines one or more properties of the target biomolecule, wherein the detection technique may be selected from fluorescence spectroscopy, for example confocal microscopy, including single wavelength or multi-wavelength confocal microscopy; scattering-based readouts, such as interferometric light scattering; or electrical readouts, such as those obtained in nanopores.

19. The device according to claim 1,
wherein the detection region measures one or more properties of the target biomolecule, including size, hydrodynamic radius, molecular weight (M_{w}) charge/ ion binding capacity, iso-electric point (pI), solubility, dipole moment and/or hydrophobicity.

20. The device according to claim 1, wherein the target biomolecule is in complex with an affinity reagent and includes or more of nucleic acids, oligonucleotides, polypeptides and peptides and fragments thereof, ribonucleoproteins, a protein-nucleic acid complex, antibodies, antibody fragments, antigen binding antibody fragments, nanoparticles, nanobodies, viruses or viral-like particles, enzymes, aptamers, affimers and other non-antibody binding proteins/molecules.

21. The device according to claim 20, wherein a further affinity reagent or reagents is/are used, such that two or more affinity reagents are used; said affinity reagents being the same or different but targeting a different epitope.

22. The device according to claim 20, wherein one or more of the affinity reagents is/are further linked to a nucleotide moiety; preferably, an oligonucleotide, such as an DNA oligonucleotide or RNA oligonucleotide, or an DNA-aptamer or RNA-aptamer; optionally wherein two or more nucleotide moieties are used, thereby enabling ligation.

23. The device according to claim 20, wherein one or more of the affinity reagents is/are further linked to a fluorophore moiety; optionally wherein two or more fluorophores are used enabling, for example, detection of co-incident light at two wavelengths or recording FRET events.

24. (canceled)

25. The device according to claim 1, wherein the flow rate within the device is configured to assist separation and/or detection; including where flow rate in the separation region is slower than the flow rate in the detection region, or where the flow rate in the separation region is faster than the flow rate in the detection region.

26. (canceled)

27. The device according to claim 1, wherein the device is configured to determine target biomolecule concentration without requiring calibration.

28-31. (canceled)

32. A method of investigating a target biomolecule comprising:
a) adding a suitable affinity reagent to a solution comprising a target biomolecule to form a fluid sample comprising bound affinity reagent-target biomolecule and unbound affinity reagent;
b) introducing said fluid sample into a microfluidic device according to claim 1;
c) separating said bound affinity reagent-target biomolecule from unbound affinity reagent target biomolecule in said separation region, wherein the separation is carried out in a continuous free flow or non-continuous batch process; and
d) performing detection on said bound affinity reagent-target biomolecule in said detection region; wherein the affinity reagent dissociation constant Kd and affinity reagent concentration are selected to ensure target biomolecule-affinity reagent binding during detection.

33-36. (canceled)

37. The method according to claim 32, wherein the detection step is carried out using continuous scanning or stepwise scanning.

38. The method according to claim 32, wherein the method uses digital counting of target biomolecules.

39-50. (canceled)

51. The method according to claim 32, wherein the affinity reagent-target biomolecule binding interaction is maintained during substantially the entire sensing process.

52-55. (canceled)

56. A method for detecting a target biomolecule biomarker, performed on a sample from a subject, such as a blood sample, tumour sample, tissue sample including brain tissue sample or other sample, said method comprising:
a) optionally carrying out processing step(s) on said subject sample to obtain a fluid sample;
b) adding a suitable affinity reagent to said fluid sample to form a fluid sample comprising bound affinity reagent-target biomolecule and unbound affinity reagent;
c) introducing said fluid sample into a microfluidic device according to claim 1;
d) separating said bound affinity reagent-target biomolecule from unbound affinity reagent target biomolecule in said separation region;
e) detecting said bound affinity reagent-target biomolecule in said detection region; such that detection of said target biomolecule according to clinically relevant parameters indicates presence of disease.

57. An in vitro method for identifying an individual having risk of disease, including but not limited to cancer, neurodegenerative diseases, infectious diseases including viral, bacterial or other pathogens, or cardiovascular disease, comprising:
a) optionally carrying out processing step(s) on a sample from a subject sample to obtain a fluid sample;
b) adding a suitable affinity reagent to said fluid sample to form a fluid sample comprising bound affinity reagent-target biomolecule and unbound affinity reagent;
c) introducing said fluid sample into a microfluidic device according to claim 1;
d) separating said bound affinity reagent-target biomolecule from unbound affinity reagent target biomolecule in said separation region;
e) detecting said bound affinity reagent-target biomolecule in said detection region; to detect an individual having risk of said disease.

58-62. (canceled)


SEQUENCE LISTING (3 sequences)

SEQ ID NO: 1 - DNA, 24 bp, Artificial Sequence
  conjugate sequence
  misc_feature LOCATION: (1)..(1): 5' Atto488 tag
  misc_feature LOCATION: (24)..(1): 3' Biotin tag
cgacatctaacctagctcactgac

SEQ ID NO: 2 - DNA, 38 bp, Artificial Sequence
  aptamer probe
  misc_feature LOCATION: (1)..(1): 5'-Atto488 tag
tggggcacgtttatccgtccctcctagtggcgtgcccc

SEQ ID NO: 3 - DNA, 28 bp, Artificial Sequence
  T-SO508 aptamer
  misc_feature LOCATION: (1)..(1): 5' Alexa488 tag
ttttgcctgtggtgttggggcgggtgcg